(19) United States
(12) Patent Application Publication
(10) Pub. No.: US 2026/0240773 A1
(43) Pub. Date: Aug. 20, 2026

(54) EXTRACELLULAR VESICLE-LIPOSOME HYBRID SYSTEM CONTAINING AN ANTIRETROVIRAL DRUG FOR THE TREATMENT OF HIV NEUROPATHOGENESIS

(71) Applicant: University of Tennessee Research Foundation, Memphis, TN (US)

(72) Inventors: Santosh Kumar, Memphis, TN (US); Harry Kochat, Memphis, TN (US); Lina Zhou, Memphis, TN (US)

(73) Assignee: University of Tennessee Research Foundation, Memphis, TN (US)

(21) Appl. No.: 19/539,518

(22) Filed: Feb. 13, 2026

Related U.S. Application Data
(60) Provisional application No. 63/758,883, filed on Feb. 14, 2025.

(51) Int. Cl. A61K 9/1271; A61K 9/00; A61K 31/12; A61K 31/47; A61K 31/635; A61K 47/24; A61K 47/28; A61P 31/18

(52) U.S. Cl. CPC A61K 9/1271; A61K 9/0043; A61K 31/12; A61K 31/47; A61K 31/635; A61K 47/24; A61K 47/28; A61P 31/18

(57) ABSTRACT

The present invention discloses an extracellular vesicle-liposome hybrid formulation for use in the treatment of HIV neuropathogenesis, including neurocognitive disorders such as HIV-associated neurocognitive disorder (HAND), which incorporates an antiretroviral therapy (ART) drug to ameliorate the problems of minimal blood-brain barrier penetration commonly associated with conventional antiretroviral drug delivery systems.

### CROSS REFERENCE TO RELATED APPLICATION

This application claims the benefit of U.S. Provisional Patent Application Ser. No. 63/758,883 filed Feb. 14, 2025, which is incorporated herein by reference.

### STATEMENT OF FEDERALLY SPONSORED RESEARCH

This invention was made with government support under grant numbers AG081140 and NM125670 awarded by the National Institute of Health (NIH). The government has certain rights in the invention.

### TECHNICAL FIELD

This invention relates generally to the field of therapeutics for treatment of diseases, and particularly diseases impacting the central nervous system (CNS), such as HIV neuropathogenesis.

This invention further relates to the field of treatment of HIV neuropathogenesis using antiretroviral therapy (ART) drugs in ways that ameliorate the problems of minimal blood-brain barrier penetration commonly associated with conventional antiretroviral drug delivery systems.

### BACKGROUND

The availability and accessibility of antiretroviral therapy (ART) have greatly increased since the 1990s, resulting in improved health outcomes and increased life expectancy for people living with human immunodeficiency virus (HIV). Despite significant advances in treatment, a complete cure for HIV has yet to be developed. HIV continues to be a major global health concern, affecting approximately 38.4 million individuals worldwide as of 2021. With the progression of HIV, 42.6% of patients are suffering from HIV-associated neurocognitive disorder (HAND) and dysfunctions such as dementia, impaired memory, and cognitive impairment.

HIV can be detected in the cerebrospinal fluid (CSF) remarkably early in the course of infection, potentially as soon as eight days after transmission. This early penetration of HIV into the central nervous system (CNS) underscores the virus's capacity to establish the infection beyond the primary immune defense systems. HIV replication within the CNS primarily occurs in perivascular macrophages and microglia, triggering a significant innate immune response. This response is characterized by the production of inflammatory factors such as cytokines and chemokines, along with oxidative stress, all of which contribute to the development of HAND. Astrocytes also play a crucial role by supporting low-level HIV replication, which allows the virus to persist and potentially establish a latent infection within the CNS. Effective suppression of HIV in the HIV reservoirs in the brain hinges on maintaining optimal concentrations of ART drugs. However, the blood-brain barrier (BBB) poses a significant challenge in this context. Delivering ART drugs to the brain is particularly challenging due to the restrictive nature of the BBB, which results in subtherapeutic drug concentrations within the CNS, enabling HIV persistence. Consequently, advanced delivery systems are imperative to facilitate efficient translocation of ART drugs across the BBB for optimal therapeutic efficacy. Furthermore, the susceptibility of the ART drugs to efflux transporters, including multi-drug resistance protein 1 (MRP1) and P-glycoprotein (P-gp), further impede their ability to penetrate the BBB. Consequently, achieving therapeutic levels of ART drugs in the brain is challenging, complicating efforts to suppress HIV replication in this critical reservoir and highlighting the need for a safer and more effective drug delivery system (DDS) to enhance drug delivery across the BBB.

Extracellular vesicles (EVs) are small, lipid bilayer-encapsulated particles released by cells into the extracellular environment and found in various biological fluids such as blood, urine, and cerebrospinal fluid. These membrane-bound particles facilitate targeted delivery of molecular cargo to their originating cells, enabling precise intercellular communication. EVs are categorized mainly into exosomes (30-150 nanometers), microvesicles (100-1,000 nanometers), and apoptotic bodies (1-5 micrometers), based on their size and origin. For brain drug delivery, EVs within 200 nm of size, are considered appropriate. However, a low percentage of encapsulation efficiency (EE %) of the EV formulation poses a challenge for in vitro and in vivo studies. Liposome is a small artificial nanoparticle, composed of at least one synthetic phospholipid bilayer. It has been approved by the FDA as a safe carrier for small molecules with sufficient EE %. In the case of doxorubicin in liposome, the EE % can be more than 90%.

Recently, to overcome the low encapsulation efficiency in EVs, an extracellular vesicle-liposome (EV-Lip) hybrid system has been developed and introduced to the field of targeted drug delivery. Small molecules and genes have been loaded in EV-liposome hybrid nanoparticles to treat bone loss, cancer, pulmonary fibrosis, etc. Other than the EV-Lip hybrid system, studies have shown that the liposome-cell membrane hybrid system can deliver cancer therapy to the target site at a relatively high EE %. One example is named Nano Cell Vesicle Technology Systems (nCVTs), which combines cell membranes from U937 monocytes with synthetic lipids to create a targeted delivery mechanism for tumor sites. By retaining essential surface proteins from U937 monocytes and incorporating liposomal elements, nCVTs enhance targeting and cellular uptake while minimizing immunogenicity. However, the EE % of the nCVT is up 20%, highlighting the need for substantial optimization to improve its EE % to proceed to the clinical setting. Researchers also strived to make synthetic or bottom-up assembled EV-like liposomes to optimize the biocompatibility of the nanoparticle. This is a promising field; however, it also requires techniques to identify the membrane components of EVs for mimicking to optimize the components of the membrane lipids and membrane protein for targeted drug delivery.

### SUMMARY

The present disclosure provides a novel hybrid extracellular vesicle-liposome (EV-LIP) nanoparticle system designed for the efficient delivery of therapeutic agents. Further, in one embodiment, this disclosure provides for the encapsulation of an antirretroviral agent (ART) from various classes including darunavir (DRV), elvitegravir (EVG), and curcumin (CUR) which represent examples from ART classes such as protease inhibitors, integrase inhibitors, and pharmacological adjuvants respectively. The disclosed EV-LIP-ART-CUR combo formulations exhibit superior physicochemical stability and high loading capacity, achieving an encapsulation efficiency of at least 58% for the antiretroviral agent and at least 64% for the adjuvant. This disclosure further demonstrates the efficacy of an Extracellular Vesicles-Liposome-Darunavir (EV-Lip-DRV) formulation for the treatment of HIV neuropathogenesis including neurocognitive disorders. The EV-Lip-DRV formulation was developed through a process involving thin-film hydration and extrusion, followed by ultrafiltration to remove unloaded DRV. The encapsulation efficiency was found to be 41.75%±²0.19%, with a particle size of ~189 nm and zeta potential of ~−8.31 mV. The hemocompatibility test confirmed the safety of the formulation for red blood cells, while drug release profiles demonstrated a sustained release of DRV within 24 h. The in vitro experiment showed that EV-Lip-DRV significantly reduces HV replication in U1 macrophages and alters the pro-inflammatory cytokine and chemokine levels. Pharmacokinetic studies in C57BL/6 mice via intranasal administration revealed significantly enhanced drug delivery in the brain relative to systemic circulation and other peripheral organs. Behavioral studies using EcoHIV-infected mice indicated significant improvements in HIV-associated impaired cognitive and motor functions when treated with the EV-Lip-DRV formulation compared to DRV alone. Furthermore, analysis of brain tissues from these mice showed significantly reduced HIV-associated inflammatory response, oxidative stress, DNA damage, and neuronal damage in EV-Lip-DRV as compared to DRV alone. Taken together, these results demonstrate that EV-Lip is a vehicle for enhancing the delivery of antiretroviral drugs to the brain, ameliorating symptoms associated with HIV neuropathogenesis and improving overall outcomes in HIV treatment

By leveraging the benefits of EVs and liposomes, disclosed within are significant improvements in drug delivery across the BBB, stability, and therapeutic outcomes. The EV-Lip-DRV formulation demonstrated reasonable stability, excellent hemocompatibility, and the ability to maintain drug release profiles. The formulation also shows the potential to reduce oxidative stress, inflammation, and neuronal damage, especially caused by HIV infection. These findings underscore the potential of EV-Lip nanoparticles for addressing the challenges of HIV neuropathogenesis and potentially other neurological disorders.

This Summary is neither intended nor should it be construed as being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and the Description of Embodiments and no limitation as to the scope of the present disclosure is intended by either the inclusion or non-inclusion of elements, components, etc. in this Summary. Additional aspects of the present disclosure will become more readily apparent from the Description of Embodiments, particularly when taken together with the drawings.

### BRIEF DESCRIPTION OF FIGURES

FIG. 1 shows the development process of an EV-Lip-DRV formulation and the characterization of the formulation's physicochemical profiles. FIG. 1 further demonstrates a process for studying the in-vitro release profiles and efficacies the EV-Lip-DRV on restricting the HIV replication in HIV-infected cells. FIG. 1 further demonstrates studies performed on the drug delivery profile in wild-type and neuropathological and behavioral effects of EV-Lip-DRV in EcoHIV mice.

FIG. 2A, FIG. 2B, FIG. 2C, FIG. 2D and FIG. 2E show the preparation of, transmission electron microscopy (TEM) images of, size and zeta potential of, percentage in size-change after storage of, and Western-blot test results of EV-Lip-DRV formulations. FIG. 2A shows a schematic diagram of the preparation of EV-Liposome-DRV (EV-Lip-DRV) formulation and hypothetical structure of EV-Lip-DRV. This diagram shows the process used to create EV-Lip hybrid systems for drug loading. The process involves integrating liposome phospholipids, EV phospholipids, cholesterol, EV membrane markers, and the drug to form the EV-Lip hybrid. The encapsulation efficiency percent (EE %) of EV-Lip-DRV is 41.75±2.19%. FIG. 2B shows transmission electron microscope (TEM) images of EVs and EV-Lip-DRV, which reveal the morphological characteristics of ECs and EV-LIP-DRV. FIG. 2C shows the size and zeta potential of EV and EV-Lip-DRV. EV: 142.90±5.26 nm, −12.47±0.38 mV; EV-Lip: 186.60±0.67 nm, −4.40±0.38 mV; EV-Lip-DRV: 189.10±2.42 nm, −7.76±0.41 mV. FIG. 2D shows the percentage of size change 29, 37, 45, 50, 78 days storage at room temperature and 4° C. after preparation (n=3), with percentage size change plotted on the dependent axis and days stored plotted on the independent axis. FIG. 2E shows the Western blot of EV and EV-Lip-DRV from three different batches. The mean±SEM provided is from n=4.

FIG. 3A, FIG. 3B, FIG. 3C and FIG. 3D collectively demonstrate the in-vitro hemocompatibility and drug release profile of EV-Lip-DRV. FIG. 3A shows several microscopic views of human red blood cells (RBCs) under exposure to dH₂O, PBS, EV-Lip, DRV, EV-Lip-DRV at indicated concentrations. FIG. 3B shows a visual inspection of RBCs after incubating with indicated solutions at 37° C. for 2 hours. FIG. 3C shows a chart comparing the ratio of hemolysis of RBCs on the dependent axis with exposure to EV-Lip-DRV formulations at indicated concentrations on the independent axis. FIG. 3D shows the in-vitro drug release profile, with drug release percentage plotted on the dependent axis and time in hours on the independent axis. The cumulative drug release was measured at 5 min, 15 min, 30 min, 1 h, 2 h, 3 h, 6 h, 9 h, 12 h, 24 h, 36 h, 48 h, 72 h, 96 h. At 24 h, 95.77% of DRV was released from the EV-Lip formulation.

FIG. 4A shows the results of U1 monocytes being differentiated into macrophages upon treating 100 nM PMA for 72 h followed by treatment of 6 μg/ml DRV, EV-Lip-DRV for 0.15, 0.5, 1, 4, 10, 24, 48, and 72 hours. FIG. 4B shows the area under the curve (AUC) estimated based on the DRV concentrations measured by LC-MS. DRV concentration was measured using per mg of protein in cell extract. (Mean±SEM, n=4).

FIG. 5A, FIG. 5B, FIG. 5C, FIG. 5D and FIG. 5E show the results of test batteries of U1 macrophages which were treated with 6 μg/ml of DRV or EV-Lip-DRV for 24 h, 48 h, and 72 h. The control group consists of U1 macrophages cultured under the same conditions but without any treatment. Media was collected for further analysis. FIG. 5A shows cytotoxicity for control, DRV, EV-Lip, and EV-Lip-DRV groups after 24 hours, 48 hours, and 72 hours. FIG. 5B shows total antioxidant compacity indicated by M of Copper Reducing Equivalents (CRE) at 24 h, 48 h, and 72 h. FIG. 5C shows reactive oxygen species (ROS) activity in the direct treatment of the U1 macrophages treated with 6 μg/ml of DRV or EV-Lip-DRV for 24 h, 48 h, and 72 h. FIG. 5D shows ROS activity measured in the direct treatment of U1 by flow cytometry using CM-DCFDA dye and excitation/emission at 495/519 nm. Data is quantified using florescent cell count that was measured in %. FIG. 5E shows the HIV p24 protein level in U1 macrophage culture media measured to indicate the level of HIV-1 replication in U1 macrophages after exposure to 6 μg/mL DRV/EV-Lip-DRV for 24 h, 48 h and 72 h. p24 level in U1 media was measured. The group without treatment is indicated as control group. Statistical analyses were carried out by using ANOVA. Results are expressed as means±SEM of n=3. *p<0.05, **p<0.01, ***p<0.001.

FIG. 6A, FIG. 6B, FIG. 6C, FIG. 6D, FIG. 6E, FIG. 6F and FIG. 6G show the cytokine and chemokine profiles in U1 macrophages treated with DRV, EV-Lip, and EV-Lip-DRV for 48 h, measured using multiplex ELISA. For pro-inflammatory cytokines, FIG. 6A for TNF-α and FIG. 6B for IL-6 show there was a significant decrease in TNF-α and IL-6 levels in both EV-Lip and EV-Lip-DRV groups compared to the control (*p<0.05, **p<0.01). For pro-inflammatory cytokine IL-1β, FIG. 6C shows that IL-1β levels were significantly lower in DRV, EV-Lip, and EV-Lip-DRV groups compared to the control (***p<0.001). For pro-inflammatory cytokine IL-18, FIG. 6D shows there was a substantial reduction in IL-18 levels in all the groups DRV, EV-Lip, and EV-Lip-DRV compared to the control (***p<0.001). Interestingly, EV-Lip also showed a decrease in these proinflammatory cytokine levels. However, there was no further decrease in these proinflammatory cytokines by EV-Lip-DRV compared to DRV or EV-Lip alone. For pro-inflammatory chemokine IL-8, FIG. 6E shows the level of IL-8 was significantly increased in EV-Lip compared to control (**p<0.01). However, its level is decreased in EV-Lip-DRV compared to EV-Lip (***p<0.001), demonstrating that the formulation decreases inflammatory response. For anti-inflammatory cytokines, FIG. 6F for IL-10 and FIG. 6G for IL-IRA show there were no significant changes in IL-10 and IL-IRA levels among the different treatment groups.

FIG. 7A shows the process and results of biodistribution of 5 mg/kg DRV administered via intraperitoneal (IP) and 2.5 mg/kg DRV using intranasal (IN) in C57BL/6 wild-type mice. FIG. 7B, FIG. 7C, FIG. 7D and FIG. 7E show that DRV concentration was determined in mice's brains (FIG. 7B), plasma (FIG. 7C), liver (FIG. 7D) and lungs (FIG. 7E), after administration of DRV or EV-Lip-DRV at 3 h. The brain-to-plasma concentration was calculated by dividing the DRV concentration in the brain by the DRV concentration in the plasma×100%. The limit of quantification (LOQ) of the LC-MS/MS methods was indicated by the dashed line. Results were expressed as means±SEM. T-tests were used to compare the differences between two treatment groups; * indicates p<0.05, *** indicates p<0.001, n=5. (also show the significance for IP vs. IN for the respective DRV or EV-Lip-DRV even though a different amount was used).

FIG. 8A, FIG. 8B, FIG. 8C, FIG. 8D, FIG. 8E, FIG. 8F, FIG. 8G, FIG. 8H, FIG. 8I and FIG. 8J show the process and results of a battery of Cognition Function and Motor Function tests. FIG. 8A shows a timeline of the behavior study of mice after HIV infection and treatment with 2.5 mg/kg of DRV or EV-Lip-DRV via IP or IN for 10 days. CatWalk and Novel Object Recognition (NOR) tests were conducted to assess the motor and neurocognitive function of mice, respectively, after HIV infection. FIG. 8B shows Heat map of NOR test. The rectangle indicates the location of the new object. FIG. 8C and FIG. 8D show the recognition index (RI) obtained before (FIG. 8C) and after (FIG. 8D) the DRV or EV-Lip-DRV treatment. FIG. 8E and FIG. 8F show the discrimination index (DI) obtained before (FIG. 8E) and after (FIG. 8F) the DRV or EV-Lip-DRV treatment. Recognition Index (RI): HIV-infected mice treated with EV-Lip-DRV via IP and IN administration showed significant improvement in RI compared to untreated HIV-infected controls (*p<0.05). However, DRV did not show a significant increase in RI using either route. Discrimination Index (DI): Both DRV and EV-Lip-DRV treatments, via IP and IN routes, showed a trend in improved DI in HIV-infected mice, aligning the DI values closer to those of the non-HIV control group but not statistically significant. FIG. 8G, FIG. 8H, FIG. 8I and FIG. 8J presents the Catwalk gait analysis results. Several parameters showed significant differences between uninfected and EcoHIV-infected and between control and treated groups in EcoHIV-infected mice. Here four parameters are presented. FIG. 8G shows that the RF Swing Speed of HIV-infected mice showed a reduced right front (RF) swing speed compared to non-HIV control. Treatment with DRV and EV-Lip-DRV via both IP and IN routes significantly improved RF swing speed, with notable increases. However, there was no difference between DRV alone and EV-Lip-DRV or between the two routes of administration. FIG. 8H shows that the RF Print Width of the HIV-infected group exhibited a narrower RF print width compared to non-HIV control. Both DRV (IP & IN) and EV-Lip-DRV (IN) treatments led to a significant increase in RF print width, indicating improved motor function. HIV-infected mice displayed altered couplings between left hind-left front (LH-LF, FIG. 8I) and right hind-right front (RH-RF, FIG. 8J) limbs, with significant deviations from the non-HIV control group. Treatments with DRV (IP) and EV-Lip-DRV (IP & IN) normalized left hind-left front (LH-LF) couplings, bringing them closer to control values with significant improvement observed, which was similar to the observations in RF Swing Speed. Only EV-Lip-DRV via IN significantly improved the coupling of right hind-right front (RH-RF) limbs.

FIG. 9A, FIG. 9B, FIG. 9C, FIG. 9D, FIG. 9E, FIG. 9F, FIG. 9G, FIG. 9H, FIG. 9I, FIG. 9J, FIG. 9K, FIG. 9L, FIG. 9M and FIG. 9N show the effect of EV-Lip DRV on neuroinflammation, neuronal damage, and oxidative stress in EcoHIV mice. FIG. 9A and FIG. 9B show HIV infection significantly elevated IL-1β (FIG. 9A) and IL-6 levels (FIG. 9B). Treatment with EV-Lip-DRV via IN administration significantly reduced IL-1β and IL-6 levels (*p<0.05). Although DRV via IP administration also reduced IL-1β levels, the change was not statistically significant. DRV via the IN route and EV-Lip-DRV via the IP route also significantly reduced IL-6 levels (*p<0.05). FIG. 9C and FIG. 9D show that no significant changes in MCP-1 (FIG. 9C) and IL-18 (FIG. 9D) levels were observed in the HIV-infected compared to the uninfected group, and among the different treatment groups compared to the non-treated group in EcoHIV. FIG. 9E shows the western blot results for representative inflammatory cytokines IL-13, IL-6, IL-18, and the chemokine MCP-1 in the brains of HIV-infected mice. FIG. 9F shows that treatment with DRV and EV-Lip-DRV via IN administration significantly reduced SOD1 levels (*p<0.05). However, FIG. 9G shows that catalase levels remained similar across all groups, demonstrating no significant changes in this oxidative stress marker. FIG. 9H shows the western blot results for oxidative stress markers, catalase, and SOD1. HIV infection increased SOD1 levels, indicating elevated oxidative stress. FIG. 9I shows that treatment with EV-Lip-DRV via both IP and IN significantly increased NeuN levels (*p<0.05). FIG. 9J shows that no significant changes were observed in IBA1 levels among the different groups. FIG. 9K shows that although synaptophysin levels were decreased in HIV-infected mice and showed an increase in the treatment groups, these changes were not statistically significant. FIG. 9L shows that no significant changes were observed in TMEM119 levels among the different groups. FIG. 9M shows the western blot results for neuronal markers (NeuN, synaptophysin), and microglia markers (IBA1 and TMEM119). HIV infection significantly reduced NeuN levels compared to non-HIV control (**p<0.01). The extent of DNA damage was evaluated by measuring 8-OHdG levels, a marker of DNA oxidation, in the brains of mice. FIG. 9N shows that treatment with EV-Lip-DRV, particularly via IP and IN administration, significantly reduced 8-OHdG levels compared to untreated HIV-infected mice (**p<0.01, ***p<0.001), indicating a reduction in DNA damage. However, no significant change was observed in DRV treatment regardless of the routes.

FIG. 10A, FIG. 10B and FIG. 10C show schematics and illustrations of EV-Lip hybrid production and fluorescence resonance energy transfer analysis for indicating membrane fusion of liposome and EV-Lip. FIG. 10A shows a schematic description of the process to produce EV-Lip hybrid to load drug. FIG. 10B shows a schematic illustration of fluorescence resonance energy transfer (FRET) membrane fusion analysis using fluorophores, rhodamine, and NBD. When lipid fusion takes place, the NBD signal increases due to the greater distance between the two probes. The FRET assay is highly sensitive to changes in the distance between fluorophores, making it an excellent tool for studying the dynamics of membrane fusion in real-time. In this study, two fluorophores were located on liposome membranes. As membranes of EV and liposomes fuse, donor (NBD) and acceptor (Rhodamine) fluorophores come further away from each other. When NBD was excited at 460 nm, it emitted light at 535 nm, which in turn excited the nearby Rhodamine. FIG. 10C shows the ratio of NBD fluorescence intensity increased in the EV-Lip hybrid, indicating the membrane fusion of liposome and EV-Lip.

### DETAILED DESCRIPTION

This disclosure provides a scalable, feasible, and cost-efficient EV-based nanoparticle drug delivery system to treat HIV neuropathogenesis including HAND. This disclosure focuses on the development of an EV-Lip hybrid nanoparticle to load an ART drug or pharmacological adjuvant to improve its distribution in the brain and ameliorate HIV neuropathogenesis including cognitive disorders. An EV-Lip-ART formulation was developed and assessed its antiretroviral efficacy in vitro, and its capability to deliver DRV to the brain and alleviate HAND-related symptoms in animal models of HIV neuropathogenesis.

EVs were isolated using a commercial kit to ensure sufficient yield within a short time frame with sufficient quality and quantity for drug formulation. A commercial kit was used to isolate EVs in order to obtain sufficient EV amount with reasonable quality for each formulation preparation from 0.5-1 ml of plasma samples. Previous trials on loading drugs in EVs using sonication and freeze-thaw method did not produce EE % higher than 12%, which is not sufficient for treatment in vitro and in vivo systems. The EE % of DRV in EV-Lip hybrid nanoparticles in this example was improved to 41.75%. The EE % of EV-Lip hybrid nanoparticles can vary widely, ranging from 15% to 90%, depending on factors such as loading methods, lipid composition, liposome/EV ratio, the physicochemical properties of the drug molecule, and the purification method used to remove unloaded drugs. For example, the doxorubicin has been loaded in different EV-Lip hybrid nanoparticles with EE % from 30% to 88%. The formulation with high EE % involves the step dissolving the EV in methanol:chloroform (1:1) and mixing with the mixed lipids solution in chloroform. Even though the EV was dissolved in the organic solvent, the EV membrane marker protein, CD63, was still detected in these hybrid nanoparticles. However, when the fusion of EV and liposome was conducted in PBS, the EE % was 30%. The preparation process can be modified without degrading the biomarker on the EV membrane to further optimize the disclosed formulation and improve the EE %.

The sizes of EV-Lip nanoparticles with and without DRV loaded were larger compared to EV. Upon fusion with liposomes, the absolute value of surface charge indicated by zeta potential was decreased. FRET analysis confirmed the membrane fusion between EV and liposome. The integrity of the vesicle upon fusion was maintained as evidenced by TEM. The membrane proteins, such as CD63, Alix, and CD9 presented on the EV membrane were confirmed to be retained in the EV-Lip hybrid nanoparticles. The EV-Lip-DRV formulation exhibited exceptional stability over time, maintaining consistent size for 78 days under both room temperature and 4° C. storage conditions. In contrast, EVs alone showed significant size changes after 45 days at room temperature and a notable increase after 78 days at 4° C., indicating their relative instability. Thus the fusion with liposomes and incorporation of DRV significantly enhances the structural stability of EVs.

While initial embodiments focus on the protease inhibitor darunavir (DRV), the platform is further optimized as an EV-LIP-ART-CUR combo system. This improved formulation incorporates the integrase inhibitor elvitegravir (EVG) co-encapsulated with curcumin (CUR). By utilizing this combination, the system significantly enhances the encapsulation efficiency and achieving approximately 58.3% for EVG and 64.6% for CUR while maintaining a stable vesicle diameter of less than 200 nm.

Oxidative stress plays a significant role in HIV pathogenesis, contributing to immune dysfunction and the progression of HAND. HIV gp120, Tat, Nef, and Vpr proteins can induce the production of ROS in microglia and astrocytes. The overproduction of ROS and depletion of antioxidants lead to cellular damage and apoptosis in neuronal cells. Studies reported that EV derived from body fluid or cells of healthy volunteers can reduce oxidative stress and suppress HIV replication. The EV is involved in HIV pathogenesis in brain, indicating the potential of using EV in the treatment of HAND. This can explain why the EV-Lip itself suppressed the HIV replication in U1 cells.

In one embodiment, the present invention provides a hybrid extracellular vesicle-liposome (EV-Lip) nanoparticle composition comprising: (i) one or more extracellular vesicles (EVs); (ii) one or more synthetic phospholipids forming a liposomal membrane component; and (iii) at least one therapeutic agent encapsulated within and/or associated with the hybrid vesicle structure.

In certain embodiments, the therapeutic agent comprises an antiretroviral therapy (ART) drug. In further embodiments, the therapeutic agent comprises a protease inhibitor, an integrase inhibitor, a reverse transcriptase inhibitor, a maturation inhibitor, an entry inhibitor, or a pharmacological adjuvant. In preferred embodiments, the ART drug is darunavir (DRV), elvitegravir (EVG), or a combination thereof. In certain embodiments, the pharmacological adjuvant is curcumin (CUR).

In one embodiment, the EV-Lip nanoparticle exhibits an encapsulation efficiency (EE %) greater than 15%. In preferred embodiments the encapsulation efficiency is at least 30%. or the encapsulation efficiency is at least 40%. In certain embodiments, co-encapsulation of an ART drug and a pharmacological adjuvant improves loading efficiency relative to EV-only formulations.

In one preferred embodiment, the hybrid nanoparticle is configured for enhanced central nervous system (CNS) delivery and is particularly useful for the treatment of HIV neuropathogenesis, including HIV-associated neurocognitive disorder (HAND).

In one embodiment, the invention provides a method of treating HIV neuropathogenesis comprising administering to a subject in need thereof a therapeutically effective amount of the EV-Lip-ART formulation.

In certain embodiments the subject is infected with HIV, the subject exhibits symptoms of HAND, the administration enhances drug delivery across the blood-brain barrier (BBB), and the brain-to-plasma drug concentration ratios are significantly increased relative to free drug administration.

In preferred embodiments, administration is via intranasal (IN) delivery, intravenous or both, or other systemic routes. In certain embodiments, intranasal administration results in enhanced brain drug levels relative to systemic circulation.

For human embodiments, the therapeutic compounds and methods are intended for use in the treatment of various medical conditions in humans. These conditions may include, but are not limited to, human immunodeficiency virus (HIV) and HIV-associated neurocognitive disorder (HAND).

Compounds described herein are synthesized using any suitable procedures starting from compounds that are available from commercial sources or are prepared using procedures described herein. General methods for the preparation of compounds as described herein are modified by the use of appropriate reagents and conditions, for the introduction of the various moieties found in the Formulas as provided herein.

In the context of the present invention, the term “patient” or “subject” refers to an individual, whether human or non-human mammal, who is receiving or undergoing treatment with the therapeutic compounds or methods disclosed herein. In human embodiments, the patient may be an individual diagnosed with a medical condition or disease for which treatment is indicated. In non-human mammalian embodiments, the subject may be an animal, such as a dog, cat, horse, rodent, or other mammalian species, for which therapeutic intervention is necessary or desirable. The terms “patient” or “subject” are used interchangeably throughout this specification to refer to the recipient of treatment, regardless of whether they are human or non-human mammals.

“Administration of” or “administering” refers to the act of delivering or applying the therapeutic compound to a patient or subject for the purpose of treating a medical condition or disease. Administration may be performed by healthcare professionals, caregivers, or the patients themselves, whether solely or under the guidance and supervision of qualified personnel.

Treating or treatment refers to the administration of a therapeutic agent to a patient with the intention of alleviating, curing, or preventing a disease or medical condition. The term may refer to any mode of therapy, including pharmacological, surgical, radiation, or other types of therapy. The term “preventing” may also encompass prophylactic treatment, which is the administration of a therapeutic agent to prevent the onset or recurrence of a disease or medical condition.

A “therapeutically effective amount” refers to a quantity of the active ingredient(s) of the compound of this disclosure that exhibits the desired therapeutic effect in a patient. The amount may vary depending on various factors, including the disease or disorder to be treated, the patient's age and health condition, the route of administration, and the desired therapeutic effect. A therapeutically effective amount may be determined by one skilled in the art using routine experimentation, and may be expressed as a range or a specific value.

The composition of the present invention can be administered through various routes to achieve therapeutic effects tailored achieve the desired therapeutic effect. Parenteral administration involves delivering the pharmaceutical composition directly into the body through means other than the digestive tract, such as subcutaneous (SC), intravenous (IV), intramuscular (IM), intraperitoneal (IP), or intrathecal routes. This route bypasses the gastrointestinal system, allowing for rapid absorption and systemic distribution of the therapeutic agent making it suitable for acute conditions or situations requiring immediate therapeutic intervention. Compositions for intravenous administration are typically in the form of sterile solutions or suspensions. Nasal administration involves delivering sprays or drops into the nasal cavity for systemic effects. Each route of administration offers unique advantages in terms of efficacy, convenience, and patient compliance, allowing for tailored treatment approaches to optimize therapeutic outcomes.

Dosage and administration regimens may vary depending on factors such as the patient's age, weight, medical condition, and response to treatment. The dosage range may be from about 10 mg to about 1000 mg per day, depending on the disease or disorder to be treated, the patient's age and health condition, and the desired therapeutic effect. The appropriate dosage and administration schedule should be determined by a qualified healthcare professional based on individual patient characteristics and therapeutic goals.

### EXAMPLES

The following Examples are presented in order to more fully illustrate the preferred embodiments of the invention. They should in no way, however, be construed as limiting the broad scope of the invention. The following methods were used to conduct the experiments described in Examples 1-10, below:

Materials: 1,2-dioleoyl-sn-glycero-3-phosphocholine (DOPC, 850375), cholesterol (700100), 16:0-06:0 NBD PC (810130), 18:1 Liss Rhod PE (810150) were purchased from Avanti Polar Lipids (Birmingham, AL, USA). DRV (TRC-D193500) was purchased from Toronto Research Chemicals, Inc. (North York, ON, Canada). The jacketed extruder (GJE-100 ml) was purchased from Genizer LLC (Los Angeles, CA, USA). Sterile phosphate-buffered saline (PBS) (10100-031) was obtained from Gibco (Dublin, Ireland). L-glutamine, penicillin-streptomycin solution, LC/MS-grade acetonitrile (A955), formic acid (AC270480010), BD PrecisionGlide 25 G needle (14-826-49), and BD 1 Ml TB syringe (14-826-88) were obtained from Fisher Scientific (Pittsburgh, PA, USA). Roswell Park Memorial Institute (RPMI) 1640 media was bought from Corning Inc. (Tewksbury, MA, USA). Fetal bovine serum (FBS) was obtained from Atlanta Biologicals (Atlanta, GA, USA). Dulbecco's modified Eagle's medium (DMEM) was obtained from the American Type Culture Collection. The immortalized mouse brain endothelial cells (bEnd.3, CRL-2299) and mouse astrocytes (C8-D1A, CRL-2541) were purchased from the American Type Culture Collection (Manassas, VA, USA). UranyLess (22409) was from Electron Microscopy Science (Hatfield, PA, USA). Constitutively HIV-infected (U1) cell lines were obtained from the NIH AIDS Reagent Program (Germantown, MD, USA). Paraformaldehyde (J19943.K2) was purchased from Thermo Fisher Scientific (Waltham, MA, USA).

### Preparation of EV-Liposomes

EVs were sourced from healthy mice plasma, and healthy human plasma, selecting based on the intended application: mice plasma-derived EVs for mice studies and human plasma-derived EVs for physicochemical characterization, U1 treatment and hemolysis studies. EVs were isolated using the total exosome isolation reagent (from plasma) according to the manual provided by the manufacturer. Briefly, the reagent is added to the plasma, and the mixture is incubated for 30 minutes at 2-8° C. The precipitated exosomes are then collected by centrifugation at 10,000×g for 5 minutes at room temperature. The resulting pellet is resuspended in 1 ml 1×PBS. Thirty milligrams of DOPC and cholesterol (70:30 mol %), 6 mg of DRV were mixed and dissolved in 4 ml of chloroform followed by a thin film of the lipids formed by rotary evaporation at 90 rpm, 40° C. for 3 h. EVs (0.6 mg based on protein amount) in PBS were added into the flask containing thin film. The mixture was swelled at 40° C. 1 h at 90 rpm. The mixture was subsequently sonicated in a water bath sonicator (Branson Ultrasonics™ 2800, Brookfield, CT, USA) for 30 s on and 30 s off for 10-15 cycles on ice. Then, the mixture was placed in the water bath at 37° C. for 30 minutes to allow the liposome to stabilize, reach equilibrium, and interact with the drug compound to enhance the encapsulation efficiency. Following this, the EV and liposome mixture was processed through an extruder for membrane fusion using a series of polycarbonate membrane filters with pore sizes of 0.4 m, and 0.2 m at room temperature in nitrogen as the air source. The mixture was processed through each membrane three times and the suspension was incubated in the water bath at 37° C. for 20 minutes to facilitate homogenization. Then, the unloaded drug was removed from the EV-Lip formulation by ultrafiltration using Amicon® Ultra Centrifugal Filter (100 kDa MWCO, Millipore Sigma, Burlington, MA, USA) according to the manufacturer's protocol.

### Determination of Morphology by Size, Zeta Potential and TEM

The average particle size and zeta potential of EV and EV-Lip nanoparticles were measured using dynamic light scattering (DLS) with a Zetasizer (Nano ZS, Malvern Instruments Inc., Malvern UK). For accurate readings, 50 μl of the formulation was diluted in 850 μl of 1×PBS (0.22 μm filtered) and placed in a disposable cuvette at room temperature. Zeta potential was calculated using the Smoluchowski equation.

The transmission electron microscope (TEM) analysis was run at 60 kV with high resolution digital camera to the morphology of the nanoparticles (JEOL-2000EX, Tokyo, Japan) by imaging center at the University of Tennessee Health Science Center (Memphis, TN). The EV and EV-Lip samples were diluted (1:500) in dH₂O (0.22 μm filtered). A 10 μl aliquot of the sample was applied to a grid, dried, and fixed with uranyless.

### Proof of Fusion Assay Using Fluorescence Resonance Energy Transfer (FRET)

Membrane fusion between EVs and liposomes was assessed using the FRET technique. Liposomes were incorporated using DOPC, cholesterol, 16:0-06:0 NBD PC, and 18:1 Liss Rhod PE in a molar ratio of 70:30:1:1 and subsequently extruded as previously described. Upon excitation at 460 nm, the emitted energy from NBD-PC at 535 nm was transferred to adjacent Rho-PE through FRET and detected at 580 nm. Fluorescence intensities at both 535 nm and 580 nm were measured simultaneously using a plate reader (Cytation 5, BioTek, Winooski, VT, USA). In this study, the two fluorophores were positioned on the liposome membranes. As the membranes of the EVs and liposomes fused, the NBD donor and Liss Rhod PE acceptor fluorophores moved apart. This spatial change resulted in increased NBD fluorescence and decreased Liss Rhod PE fluorescence, thereby confirming membrane fusion. The absorbance of H₂O at 585 nm and 535 nm was used as background. The NBD fluorescence percentage relative to the total absorbance for each sample at 585 nm and 535 nm was then calculated.

### Quantification of DRV Concentrations

The EV-Lip-DRV formulation was extracted with 10-times acetonitrile to determine the encapsulation efficiency and the measurement of DRV amount in the cell, plasma, and tissue samples using the LC-MS/MS method. The percent of encapsulation efficiency (EE %) was calculated by the following equation:

$$Encapsulation efficiency ( \% ) = \frac{Weight of the drug in nanoparticles}{Weight of the feeding drugs} \times 100 \%$$

### Measurement of In Vitro Drug Release

The in vitro release profile of EV-Lip-DRV was studied. Briefly, a total of 5 ml of the EV-Lip-DRV formulation was added to a Float-A-Lyzer® dialysis device (8-10 kDa, catalog number Z726508, Sigma-Aldrich, St. Louis, MO, USA). The dialysis device was immersed in 125 ml 1×PBS at 37° C. Samples of 20 μl from the reservoir were collected at 5, 15 and 30 min, and 1, 2, 3, 6, 9, 12, 24, 36, 48, 72, and 96 h. EV-Liposomes. The 1×PBS was replaced every 24 hours to maintain the sink condition. The samples collected were spiked in acetonitrile to measure the concentration of DRV by LC-MS/MS.

### Cell Culture

U1 cells were maintained in RPMI1640 medium prepared with 10% fetal bovine serum (FBS) and 1% L-glutamine. To induce differentiation into macrophages, 0.8 million U1 cells were suspended in 1.5 ml of medium containing 100 nM phorbol 12-myristate 13-acetate (PMA) and plated in 6-well plates. Following a 3-day differentiation period, the medium was removed, and the cells were rinsed with PBS. Then, fresh medium was then added to U1 macrophages, which were incubated for an additional 3-4 h before the treatment.

### LDH Cell Cytotoxicity Assay

Following a daily dose of 6 μg/ml of DRV or EV-Lip-DRV in U1 macrophages, cytotoxicity levels were assessed after 24, 48, and 72 h of treatment using the Pierce Lactate Dehydrogenase (LDH) Cytotoxicity Assay Kit (ThermoFisher Scientific, Grand Island, NY, USA), following the manufacturer's instructions. Briefly, the level of LDH in the cell culture media indicates cell damage and lysis. 50 μl of the media sample was combined with 50 μl of the LDH assay reagent in a 96-well plate. After 30 min incubation at room temperature, the reaction was stopped by LDH stop solution. Then the absorbance was measured at 490 nm and 680 nm using a microplate reader (Cytation 5, BioTek, Winooski, VT, USA). The cytotoxicity level was calculated by subtracting the absorbance at 680 nm from the absorbance at 490 nm.

$$Corrected LDH Activity = A 490 - A 680$$

$$Relative LDH Activity = \frac{Corrected LDH Activity of Experimental Group}{Corrected LDH Activity of Control Group} \times 100 \%$$

### Measurement of Total Antioxidant Capacity (TAC)

The antioxidant capacity of U1 macrophages following treatments was assessed using the OxiSelect™ Total Antioxidant Capacity (TAC) Assay Kit (Cell Biolabs, San Diego, CA, USA). U1 macrophages were treated with a daily dose of 6 μg/ml of DRV or EV-Lip-DRV and cell media were prepared as per the kit instructions. Samples, standards, and controls were added to a 96-well microtiter plate in 20 μl aliquots. Each well then received 180 μl of 1× Reaction Buffer. After mixing thoroughly, the initial absorbance was measured at 490 nm. The reaction was initiated by adding 50 μl of 1× Copper Ion Reagent to each well and incubating for 5 min on an orbital shaker. The reaction was terminated by adding 50 μL of 1× Stop Solution, and the final absorbance was measured at 490 nm. Antioxidant capacity was quantified by comparing the net absorbance values (final minus initial) to a standard curve generated from known concentrations of uric acid. Results were expressed as M Copper Reducing Equivalents (CRE), indicating the total antioxidant capacity of the treated U1 macrophages.

### Determination of HIV p24 by ELISA

The p24 antigen levels in U1 macrophage media were measured using the HIV p24 Antigen ELISA Kit (Zeptometrix Corporation, Buffalo, NY, USA). U1 cell media samples were collected, stored at −80° C., and thawed before analysis. Aliquots of 200 μl of standards, controls, and samples were added to a 96-well ELISA plate and incubated at 37° C. overnight. Wells were washed five times with the wash buffer, followed by the addition of 100 μl detector antibody solution and another 1-h incubation at 37° C. After five additional washes, 100 μl of Streptavidin-Peroxidase Working Solution was added and the plate was incubated at 37° C. for 30 min followed by the addition of the substrate solution, and the plate was incubated in the dark at room temperature for 20-30 min. The reaction was stopped by adding 100 μl stop solution, and absorbance was measured at 450 nm using a Cytation 5 Cell Imaging Multi-Mode Reader (BioTek, VT, USA). p24 concentrations were calculated by comparing absorbance values to a standard curve, with data corrected for any dilution factors, providing accurate quantification of p24 levels in the samples.

### Measurement of Intracellular Reactive Oxygen Species (ROS)

After daily treatment in U1 macrophage, the reactive oxygen species (ROS) levels in treated cells were quantified using flow cytometry with the fluorescent dye CM-H2DCFDA (General Oxidative Stress Indicator, Thermo Fisher Scientific, Waltham, MA, USA). Following treatment, cells were thoroughly washed with PBS to remove any residual treatment compounds. The cells were then resuspended in a solution containing 5 M CM-H2DCFDA in PBS and incubated in the dark at room temperature for 45 min to allow for dye uptake and conversion. Post-incubation, the cells were washed again with PBS to remove excess dye and resuspended in 300 μl of PBS. The fluorescence intensity, corresponding to the ROS levels, was measured using an Agilent NovoCyte flow cytometer, and data acquisition was performed with the instrument's integrated software. This method allowed for the precise detection and quantification of ROS generated by the treated cells.

### Quantification of Cellular Cytokines and Chemokines Level

To measure the protein levels of cytokines and chemokines the Human Custom ProcartaPlex 9-plex Assay Kit (ThermoFisher Scientific, Waltham, MA, USA) was employed. Cell culture supernatant samples were collected and stored at −80° C. until analysis. The assay was conducted following the manufacturer's protocol. Briefly, samples were thawed and centrifuged at 10,000×g for 5-10 min to remove particulates. The assay plate was prepared by adding 50 μl of magnetic bead solution to each well, followed by washing to remove any unbound beads. Next, 50 μl of standards, controls, and samples were added to the appropriate wells, and the plate was incubated overnight at 4° C. After incubation, the wells were washed, and 25 μl of detection antibody mixture was added to each well. The plate was incubated with shaking at room temperature for 30 min, washed again, and then 50 μl of SAPE solution was added. Following a final incubation and wash, 120 μl of reading buffer was added to each well. The plate was then read using a Luminex™ instrument to quantify the levels of each target protein.

### Measurement of Intracellular DRV Concentrations

For intracellular DRV estimation, U1 macrophages were treated with 6 μg/ml DRV/EV-Lip-DRV for 0.15, 0.5, 1, 4, 10, 24, 48, and 72 h. Cells were harvested in RIPA and were subjected to LC-MS/MS for DRV amount estimation.

### In Vitro Hemocompatibility

Red blood cells (RBCs) were isolated from a blood sample acquired from Interstate Blood Bank Inc. (Memphis, TN). This sample was collected from de-identified human donors with the approval of the Institutional Review Board at the University of Tennessee Health Science Center (UTHSC). Modifications were made to an existing protocol to conduct the hemolysis assay. A suspension of 13.5 million RBCs in 300 μl of PBS was incubated with DRV or EV-Lip-DRV formulation at concentrations of 6, 24, 60, and 120 μg/ml. This incubation was carried out for 2 h at 37° C. PBS served as the negative control, while deionized water (dH₂O) was used as the positive control. Following the incubation, the suspension was centrifuged at 1000×g for 10 min at 4° C. From this, 200 μl of the supernatant was transferred to a 96-well plate to measure the absorbance at 570 nm. The supernatant was also diluted 50× in PBS to be imaged on the glass side using Olympus IX73 inverted microscope (Evident Scientific, Inc., Waltham, MA, USA). The percentage of hemolysis was calculated using the following equation:

$$\% of hemolysis \% = \frac{[ {OD}_{test} ] - [ {OD}_{neg} ]}{[ ODpos ] - [ {OD}_{neg} ]} \times 10$$

### Animal Study:

### Drug Delivery to Mice Brain

Ten to twelve-week-old male and female C57BL/6 mice were obtained from Jackson Laboratory (Bar Harbor, MA). Upon arrival, the mice were acclimated to the animal facility for a minimum of seven days. The animals were housed in groups of five per cage in a sterile room with a 12-h light/dark cycle. The room temperature and humidity were consistently regulated. Food and water were provided ad libitum. Animal studies were conducted under the protocol approved by the Institutional Animal Care and Use Committee (IACUC) at UTHSC. For the investigation of the permeability of the EV-Lip-DRV, the DRV or EV-Lip-DRV was dosed via intraperitoneal (IP) and intranasal (IN) routes. For IN, the maximum volume used for one nostril is 100 μl. (n=5, IP: 5 mg/kg, IN: 2.5 mg/kg) DRV for the free drug group was prepared in 5% DMSO, 80% PEG400, and 15% PBS. The brain, lungs, and liver were collected at 3 h after administration. Due to the limited volume that can be dosed via the IN route, mice were dosed with a 30-minute gap with a maximum volume of 50 μl at a time. The organs were homogenized and subjected to drug estimation using LC-MS/MS.

### Infection of EcoHIV in Mice

The chimeric HIV, EcoHIV-NDK, was used to infect the C57BL/6J mice following the protocol published by Alfar et al. EcoHIV-NDK plasmid was acquired from Volsky lab at the School of Medicine at Mount Sinai. Briefly, EcoHIV-NDK virus stocks were generated by transfecting plasmid DNA into 293T cells. Cell media was collected, and the virus amount was determined by the p24 level following the aforementioned ELISA method. The virus stock was stored at −80° C. and resuspended in saline for retro-orbital injection. Each mouse was injected with 8.4 μg of virus as of p24 level. After 21 days of EcoHIV infection, 2.5 mg/kg of DRV or EV-Lip-DRV were administered to mice daily via IN or IP routes. Behavior studies were conducted on day 20 and day 31. Mice were euthanized on day 31 and brains were collected for further pathological analysis.

### The Novel Object Recognition (NOR) Test

The NOR test was conducted following the published method⁸⁴. During the training session, mice were habituated to an empty arena, consisting of a standard-size box (45×45×35 cm), for 10 min each day over 5 consecutive days. In the testing session, each mouse was presented with two identical objects during the first session for 10 min. After the sample object exposure, the animal was returned to its home cage for a 1-h retention period. In a second session, one of the objects was replaced with a new object for 5 min. The amount of time the mice spent exploring the new object served as an index of recognition memory. The arena and objects were cleaned with 70% ethanol after each session. All trials were recorded, and video tracked using the Etho-Vision XT 7 automated tracking system (Noldus, Leesburg, VA, USA).

The software computed the time spent exploring each object during the test sessions. This data was then used to determine the recognition index (RI), and discrimination index (DI) calculated with the following equations:

$$Discrimination index ( DI ) = \frac{Novel object Exploration - Familiar Object Exploration Time}{Total Exploration Time}$$

$$Recognition index ( RI ) = \frac{Novel Object Exploration Time}{Total Exploration Time}$$

### CatWalk XT—Gait Analysis

The catwalk test for gait analysis was conducted using the CatWalk XT automated analysis apparatus (Noldus Information Technology, Wageningen, The Netherlands) on Day 31. A detailed description of the gait analysis has been previously published⁸⁵. In brief, the mice were trained to cross the walkway for 30-45 min each day over six consecutive days before establishing the baseline. The CatWalk system consists of a glass-surfaced walkway illuminated with a green light from below and a red light source from above. The CatWalk XT system captures images of the animal's footprints and foot force profiles as they cross the illuminated glass plate. The green light highlights the paw prints, while the red light provides contrast for the animal's body contour, recorded by an automated camera below the glass plate. On the final day of the experiment, each animal underwent three trials, and the mean values from these trials were used for the final analysis.

### Western Blot

The protein expression was assessed related to inflammatory cytokines and chemokines, oxidative stress markers, and neuronal damage markers in the control and EcoHIV mice brains using Western blotting. The mice brain samples were homogenized in three times the volume of PBS. For BCA analysis, brain homogenates were diluted in two times the volume of RIPA. After 10-15 min incubation on ice, samples were subjected to 10,000×g centrifugation for 5 min. The supernatant was collected and diluted six times in RIPA for the measurement of the protein concentration of the brain samples.

The protein expression of inflammatory cytokines and chemokines, oxidative stress markers, and neuronal damage markers in the control and EcoHIV mouse brains w evaluated using Western blotting. Mouse brain samples were homogenized in three volumes of PBS. For BCA analysis, brain homogenates were diluted with two volumes of RIPA buffer. After incubating on ice for 10-15 min, the samples were centrifuged at 10,000×g for 5 min. The supernatant was collected and diluted six-fold in RIPA buffer to measure the protein concentration using the Pierce™ BCA Protein Assay Kit (23225, Thermo Scientific, Waltham, MA, USA).

The expression of inflammatory cytokines and chemokines, including IL-6, IL-1β, MCP-1, and IL-18, along with oxidative stress markers superoxide dismutase 1 (SOD1) and catalase were assessed. Neural marker proteins NeuN, IBA1, Synaptophysin, and TMEM119 were used to evaluate neural damage. All protein expression levels were normalized to β-actin. Equal amounts of protein (12 g) from control, HIV without treatment, and HIV treated with DRV and EV-Lip-DRV mouse brain homogenates were used. Proteins were separated on a polyacrylamide gel with a 4% stacking fgel and a 10% resolving gel. Electrophoresis was conducted at 60V for 50 min, followed by 150V for another 50 min. Proteins were then transferred to a polyvinylidene fluoride (PVDF) membrane at 0.35 Amp for 90 min. Following the transfer, the membrane was incubated with 5-10 ml of Li-Cor blocking buffer (LI-COR Biosciences, Lincoln, NE, USA) for one hour to minimize nonspecific antibody binding. The membrane was then incubated overnight at 4° C. with primary antibodies specific to the target proteins. The detailed information of the primary antibodies used is summarized in Table 1.

TABLE 1
List of detailed information on the primary antibodies

| | | Dilution |
|---|---|---|
| Antibody | Species | factor |
| β-actin | mouse | 10000 |
| IL-6 | rabbit | 1000 |
| IL-1β | rabbit | 1000 |
| MCP-1 | mouse | 500 |
| IL-18 | rabbit | 1000 |
| SOD1 | mouse | 500 |
| Catalase | mouse | 1000 |
| NeuN | rabbit | 1000 |
| IBA1 | rabbit | 500 |
| Synaptophysin | mouse | 2000 |
| TMEM119 | rabbit | 500 |

The next day, the blots were washed three times with 0.2% Tween-20 PBST solution and then incubated with the appropriate secondary antibodies—Goat anti-Mouse Mab (1:10,000 dilution, LI-COR Biosciences) and Goat anti-Rabbit Mab (1:10,000 dilution, LI-COR Biosciences)—for one hour at room temperature in the dark. After an additional round of washing with PBST, the membranes were scanned using Image Studio Lite version 4.0 on a Li-Cor Scanner (LI-COR Biosciences). LI-COR Image Studio Lite version 4.0. (Nebraska, USA) software was used to perform the densitometry analyses of the proteins with β-Actin serving as an internal loading control to normalize protein expression levels.

### DNA Damage in the Brain

DNA was isolated from 100 μl brain homogenates using the QIAamp DNA mini kit (QIAGEN, Germantown, MD, USA) following the manufacturer's protocol. DNA concentrations were determined by measuring the absorbance of 1 μl of the sample at 260/280 nm with a NanoDrop™ 2000 spectrophotometer (Thermo Scientific, Waltham, MA, USA).

### Example 1

### Development and Characterization of EV-Lip-DRV Formulation

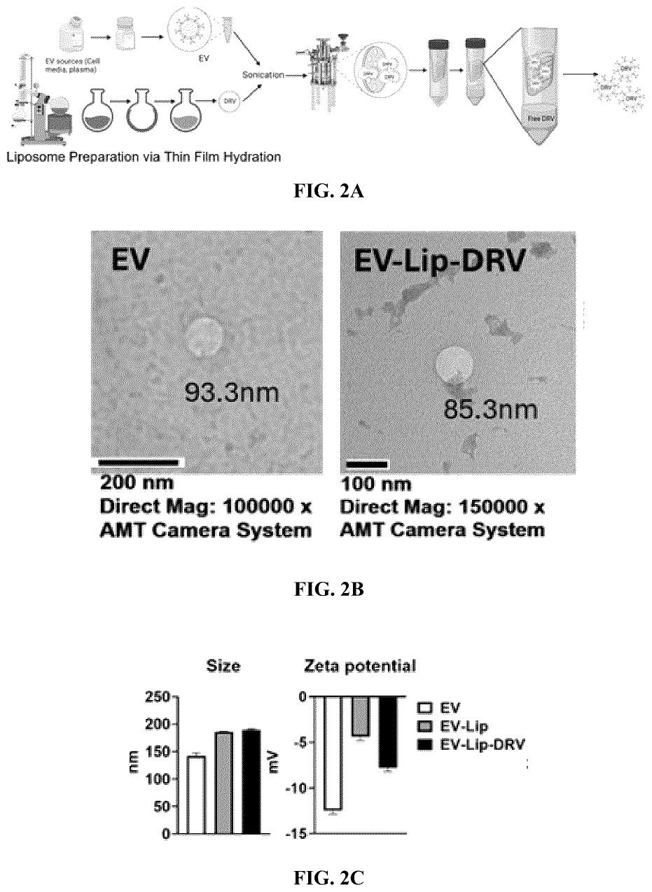

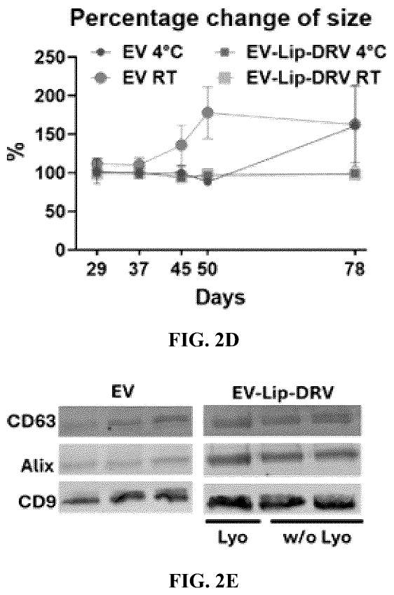

A schematic representation of the preparation of EV-Liposome-DRV (EV-Lip-DRV) is illustrated in FIG. 2A. This process involves the use of EV sources from cell media or plasma, followed by thin film hydration to incorporate DRV into liposomes, sonication of the EV and liposomes, and extrusion, resulting in the EV-Lip-DRV formulation. The free DRV or unloaded DRV was removed from the formulation by ultrafiltration. The ratio of the lipids and drug was optimized to ensure the adequate concentration of DRV in the formulation to proceed with the animal studies. The encapsulation efficiency percent (EE %) of the formulation from eight preparations was 41.75%±2.19%.

A schematic representation of the preparation of EV-Liposome-DRV (EV-Lip-DRV) is illustrated in FIG. 2A. This process involves the use of EV sources from cell media or plasma, followed by thin film hydration to incorporate DRV into liposomes, sonication of the EV and liposomes, and extrusion, resulting in the EV-Lip-DRV formulation. The free DRV or unloaded DRV was removed from the formulation by ultrafiltration. The ratio of the lipids and drug was optimized to ensure the adequate concentration of DRV in the formulation to proceed with the animal studies. The encapsulation efficiency percent (EE %) of the formulation from eight preparations was 41.75%±2.19%.

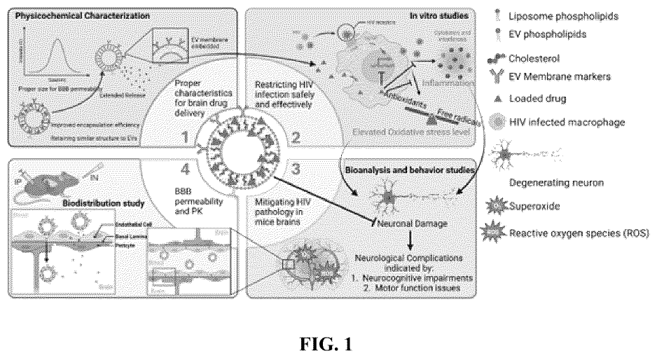

TEM images in FIG. 2B revealed the morphological characteristics of EVs and EV-Lip-DRV. The EVs displayed a size of approximately 93.3 nm, while the EV-Lip-DRV exhibited a slightly reduced size of 85.3 nm. The size and zeta potential of EV, EV-Lip, and EV-Lip-DRV formulations were measured and compared (FIG. 1C). The average size of the EVs was found to be 142.90±5.26 nm, with a zeta potential of 12.47±0.38 mV. In contrast, the EV-Lip formulation exhibited an increased size of 186.60±0.67 nm and a reduced zeta potential of 4.40±0.38 mV. The EV-Lip-DRV formulation showed a similar size to EV-Lip at 189.10±2.42 nm and a zeta potential of 7.76±0.41 mV. These results indicate that the encapsulation of DRV during the fusion of EVs and liposome membranes led to a slight increase in particle size and a modification in surface charge. The changes in size were measured on days 29, 37, 45, 50, and 78 following the isolation of EVs or the preparation of EV-Lip-DRV (FIG. 2D). The size of EVs changed significantly after 45 days at room temperature and increased markedly after 78 days when stored at 4° C. In contrast, the EV-Lip-DRV maintained its size consistently over the 78 days, regardless of the storage conditions. Western blot analysis, as shown in FIG. 2E, confirms the presence of EV markers CD63, Alix, and CD9 in both EVs and EV-Lip-DRV. The consistency of marker expression across three different batches demonstrates the reproducibility of the formulation process. The retention of plasma EV-specific markers on EV-Lip-DRV membranes enhances the potential for effective drug delivery to the brain by improving targeting, biocompatibility, stability, cellular uptake, and controlled release of the drug.

### Example 2

### Hemocompatibility and Drug Release Profile of EV-Lip-DRV

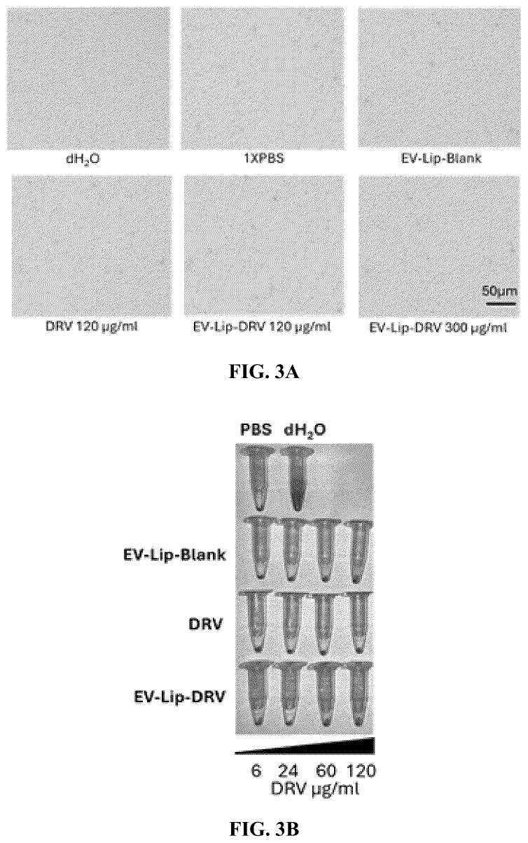

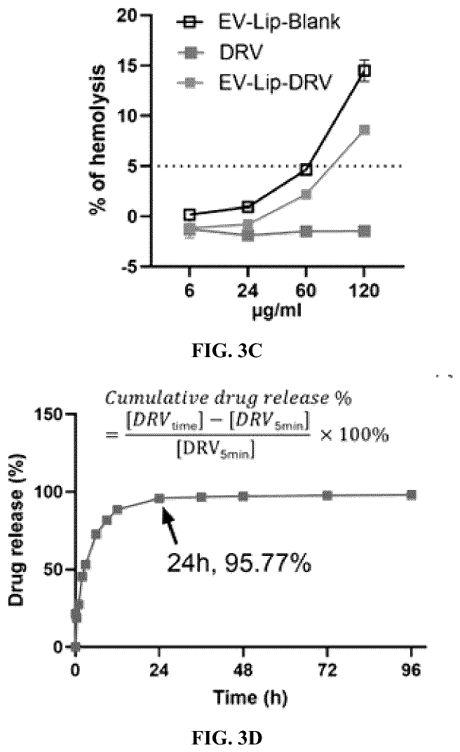

FIG. 3A presents microscopic images of human RBCs exposed to various solutions (dH₂O, PBS, EV-Lip-Blank, DRV at 120 μg/ml, and EV-Lip-DRV at 120 μg/ml, and EV-Lip-DRV at 300 μg/ml) following a 2-h incubation at 37° C. The images show that RBCs exposed to EV-Lip-DRV formulations maintained their morphology without significant changes compared to the control groups, indicating good hemocompatibility. Visual inspection of RBCs in FIG. 3B corroborates these findings, with RBCs treated with dH₂O (positive control) displaying significant hemolysis, as evidenced by the red coloration, whereas EV-Lip-DRV formulations caused minimal hemolysis, comparable to the PBS and EV-Lip-Blank controls. Further quantification in FIG. 3C shows the hemolysis percentage for RBCs exposed to different concentrations (6, 24, 60, 120 μg/ml) of EV-Lip-Blank, DRV, and EV-Lip-DRV, revealing that the hemolysis percentage for EV-Lip-DRV remained consistently below 15%, which is within the acceptable range for hemocompatibility. This confirms that the EV-Lip-DRV formulations are safe for RBCs. The microscopic images, visual inspection images, and hemolysis data indicate that EV-Lip-DRV formulations exhibit good hemocompatibility. The percentage of hemolysis remains relatively low across different concentrations (6, 24, 60 μg/mL), demonstrates that the formulation is safe for even intravenous administration.

FIG. 3D presents the cumulative drug release profile of DRV from the EV-Lip formulation. The release was monitored at various time points (5 min, 15 min, 30 min, 1 h, 2 h, 3 h, 6 h, 9 h, 12 h, 24 h, 36 h, 48 h, 72 h, 96 h). The results showed that 95.77% of DRV was released from the EV-Lip formulation within 24 h, demonstrating a sustained release profile. The cumulative drug release profile showed an extended release for 48 h. This demonstrates that the formulation is efficient in delivering the drug in an extended-release manner, which is critical for therapeutic efficacy. These findings demonstrate the impact of modifying the components of the hybrid nanoparticle system on the drug release profile and highlight the influence of pH on drug release kinetics. Such modifications can be strategically employed to tailor the EV-Lip nanoparticle system for specific therapeutic needs, optimizing the release rate for extended or controlled delivery.

### Example 3

### Effective Uptake of EV-Lip-DRV in Differentiated Macrophages

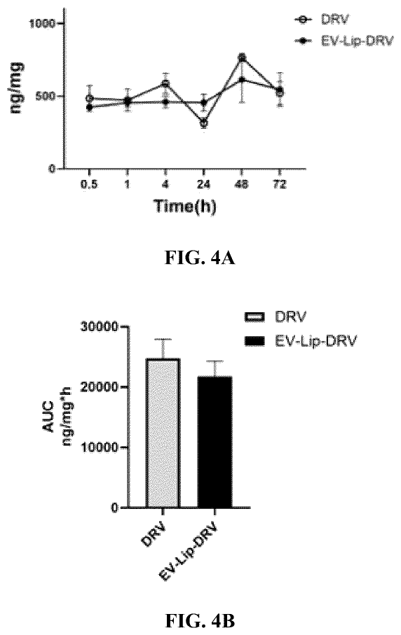

As shown in FIG. 4A and FIG. 4B, the concentration of DRV in U1 macrophages was measured at indicated time points using LC-MS, and the data were used to calculate the area under the curve (AUC). The results demonstrate that DRV and EV-Lip-DRV have similar drug concentration profiles over time, with no significant differences observed in the AUC between the two groups. This demonstrates that the encapsulation of DRV in EV-Lip does not adversely affect the drug's uptake and retention in macrophages. The comparable AUC values further confirm the delivery and retention of DRV from the EV-Lip-DRV formulation in U1 macrophages.

The cellular uptake of the drug in the hybrid nanoparticle can vary with certain EV:Lipid ratios indicated by the protein of EV and weight of lipids. The EV:Lipid ratio in this example is 1:50. These data demonstrate that encapsulation of DRV in EV-Lip with a 1:50 EV:Lipid ratio does not negatively impact its uptake and retention in U1 macrophages. The increase of EV does not necessarily assure advanced cellular uptake, such as the pancreatic cancer cell line derived EV showed lower cellular uptake compared to its EV-Lip nanoparticle. The EV-Lip-Doxorubicin in this study showed significant suppressive effect in the migration test of three cell lines used in this work. The EV-Lip nanoparticle loaded with clodronate prepared using fibroblast cell line (L-929) derived EVs, demonstrated improved cellular uptake in L-929 cells compared to its liposome formulation but not in the macrophage cell line (RAW 264.7). However, the EV-Lip loaded with clodronate showed enhanced therapeutic efficacy for pulmonary fibrosis treatment in a pulmonary fibrosis mice model.

### Example 4

### Effect of EV-Lip-DRV on Cytotoxicity, Antioxidant Capacity and Reactive Oxygen Species (ROS) Activity in U1 Macrophages

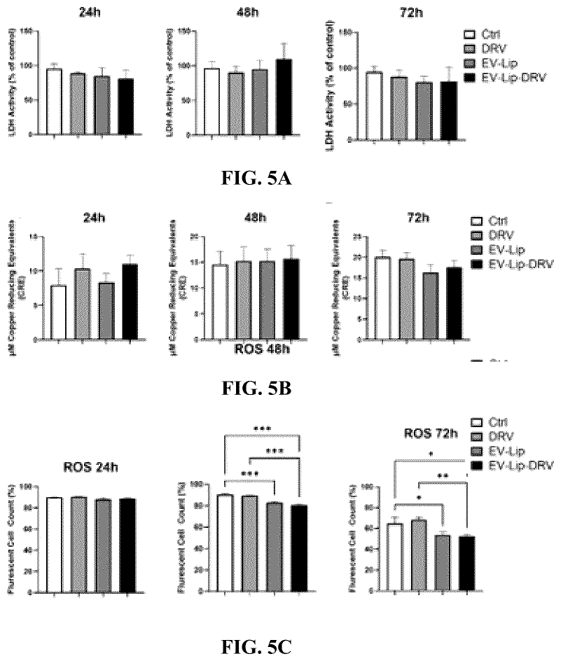

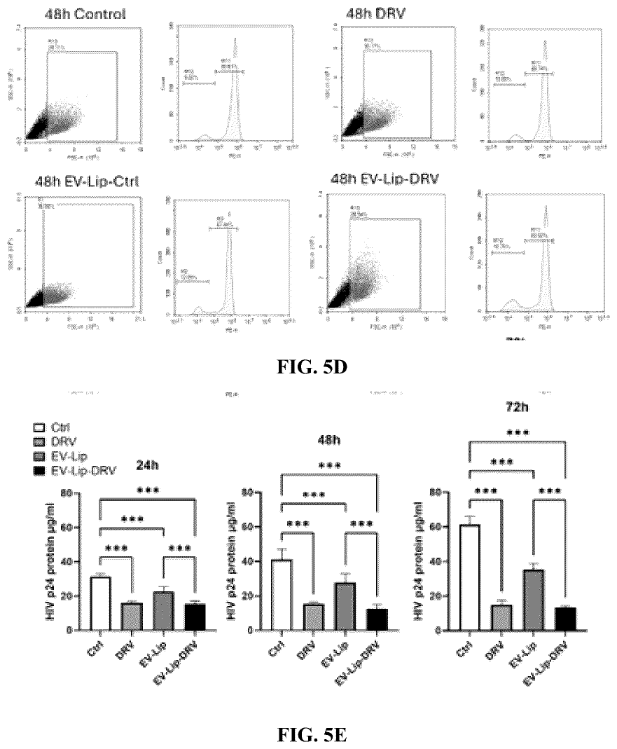

As shown in FIG. 5A, lactate dehydrogenase (LDH) activity was measured to assess cytotoxicity. There were no significant differences in LDH activity across all groups (Ctrl, DRV, EV-Lip, EV-Lip-DRV) at 24 h, 48 h, and 72 h, indicating that the treatments did not induce cytotoxicity.

FIG. 5B presents the total antioxidant capacity of the cells, measured in micromolar copper-reducing equivalents (CRE). The antioxidant capacity remained stable across all treatment groups at 24 h, 48 h, and 72 h, demonstrating that neither DRV nor EV-Lip-DRV treatments adversely affected the cells' antioxidant defenses.

FIG. 5C and FIG. 5D showed the measurement of ROS activity through flow cytometry using CM-DCFDA dye. At 24 h, there was no significant difference in ROS levels among the groups. However, at 48 h, the fluorescent cell count, indicative of ROS activity, showed a significant reduction in the EV-Lip groups compared to the HIV control group (***p<0.001). At 72 h, ROS levels significantly decreased in both the EV-Lip and EV-Lip-DRV treatment groups compared to the HIV control group (*p<0.05, **p<0.01, ***p<0.001). Specifically, the EV-Lip-DRV group maintained a higher fluorescent cell count, aligning with the quantified ROS data. This indicates that the effect of the EV-Lip treatments was sustained throughout the 72-h period.

Overall, the results demonstrate that DRV and EV-Lip-DRV treatments do not induce cytotoxicity or significantly alter antioxidant capacity in U1 macrophages. However, ROS activity was modulated over time, with significant reductions observed at 48 h and 72 h, particularly in the DRV and EV-Lip groups. This demonstrates that while the EV-Lip-DRV formulation is effective in reducing oxidative stress, it maintains better ROS stability over time compared to DRV and EV-Lip alone.

FIG. 5E illustrates the levels of HIV replication in U1 macrophages after 24 h, 48 h, and 72 h of exposure with 6 μg/ml DRV or EV-Lip-DRV. HIV replication was assessed by measuring p24 protein levels in the U1 media. At all the time points, both DRV and EV-Lip-DRV significantly reduced p24 levels compared to the control group (***p<0.001). However, compared to DRV alone, there was no further decrease in p24 level by EV-Lip-DRV treatment. Compared to control, EV-Lip also showed a decrease in the viral load. These results demonstrate at least EV-Lip-DRV is as effective as DRV alone in reducing HIV replication in U1 macrophages.

This demonstrates that the EV-Lip-DRV formulation significantly inhibits HIV replication in U1 macrophages, as evidenced by the reduction in p24 levels at 24, 48, and 72 hours without inducing cytotoxicity and reducing antioxidant capacity.

### Example 5

### EV-Lip-DRV Alters HIV-Associated Inflammatory Response

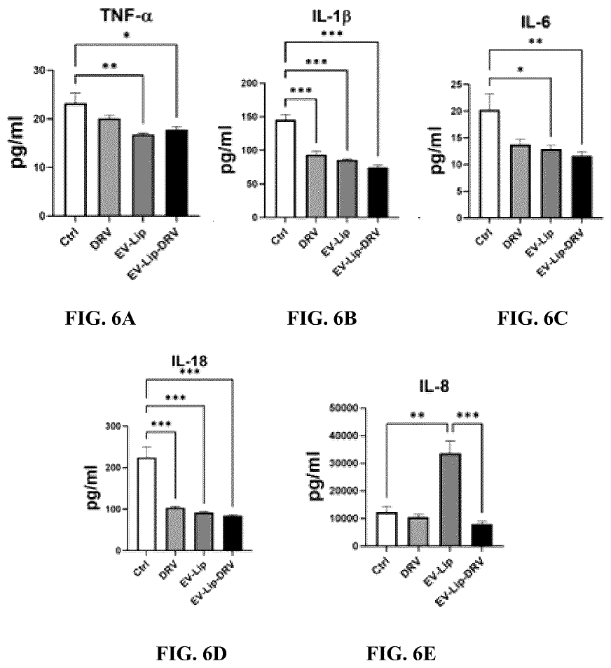

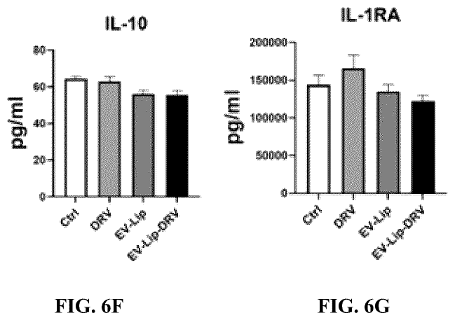

FIG. 6A, FIG. 6B, FIG. 6C, FIG. 6D, FIG. 6E, FIG. 6F and FIG. 6G show the cytokine and chemokine profiles in U1 macrophages treated with DRV, EV-Lip, and EV-Lip-DRV for 48 h, measured using multiplex ELISA. Pro-inflammatory Cytokines TNF-α (FIG. 6A) and IL-6 (FIG. 6C): There was a significant decrease in TNF-α and IL-6 levels in both EV-Lip and EV-Lip-DRV groups compared to the control (*p<0.05, **p<0.01). IL-1β (FIG. 6B): IL-1β levels were significantly lower in DRV, EV-Lip, and EV-Lip-DRV groups compared to the control (***p<0.001). IL-18 (FIG. 6D): There was a substantial reduction in IL-18 levels in all the groups DRV, EV-Lip, and EV-Lip-DRV compared to the control (***p<0.001). Interestingly, EV-Lip also showed a decrease in these proinflammatory cytokine levels. However, there was no further decrease in these proinflammatory cytokines by EV-Lip-DRV compared to DRV or EV-Lip alone. Pro-inflammatory Chemokine IL-8 (FIG. 6E): The level of IL-8 was significantly increased in EV-Lip compared to control (**p<0.01). However, its level is decreased in EV-Lip-DRV compared to EV-Lip (***p<0.001), demonstrating that the EV-Lip-DRV formulation decreases inflammatory response. Anti-inflammatory Cytokines IL-10 (FIG. 6F) and IL-1RA (FIG. 6G): There were no significant changes in IL-10 and IL-IRA levels among the different treatment groups.

Overall, both EV-Lip and EV-Lip-DRV treatment modulates the cytokine and chemokine profile, significantly reducing the levels of several pro-inflammatory cytokines (TNF-α, IL-1β, IL-6, IL-18), while not affecting the anti-inflammatory cytokines (IL-10 and IL-1RA). However, a decrease in pro-inflammatory cytokine IL-8 by EV-Lip-DRV, but not by EV-Lip formulation demonstrates that the drug-encapsulated formulation may reduce inflammatory response in U1 macrophages. Taken together, EV-Lip-DRV formulation not only reduces the oxidative stress but also reduces the inflammatory response in U1 macrophages.

### Example 6

### Permeability of DRV and EV-Lip-DRV in C57BL/6 Mice Brains

The biodistribution and pharmacokinetics of DRV and EV-Lip-DRV were evaluated in C57BL/6 wild-type mice following intraperitoneal (IP) and intranasal (IN) administration. Mice were administered 5 mg/kg DRV via IP or 2.5 mg/kg DRV via IN. DRV concentrations were measured in the brain, plasma, liver, and lungs after 3-h post-administration using LC-MS/MS. Three hours as the terminal time point was selected based on prior studies that demonstrated the detectable DRV concentrations in mice brains after 1 hour and 3 hour of IN administration. To ensure all of the brain samples were collected as the 3 hour time point, 3 hours was selected as the terminal time point in the study.

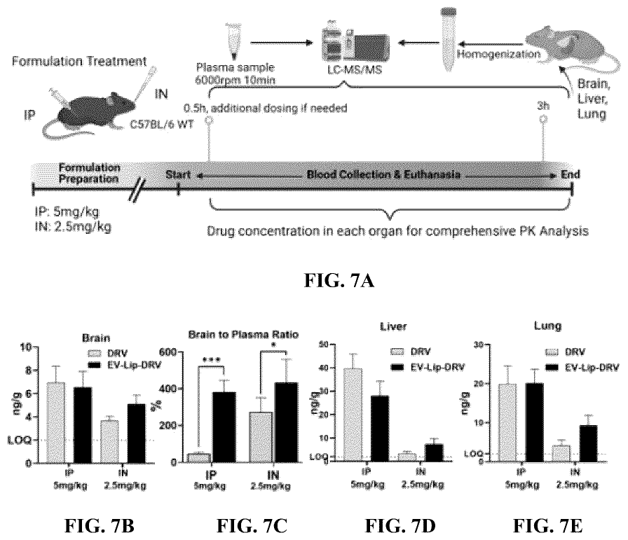

As shown in FIG. 7B, FIG. 7C, FIG. 7D and FIG. 7E, the concentration of DRV in the brain after the indicated treatment remained unchanged via IP administration. While there was an observed increase in brain DRV concentration in the EV-Lip-DRV group using the IN route, the variability in the data rendered these changes statistically insignificant. The brain-to-plasma ratio of DRV was calculated to assess the efficiency of brain targeting. The ratio was significantly higher for the EV-Lip-DRV group compared to the DRV group after both IP (***p<0.001) and IN administration (*p<0.05). These findings demonstrate that the EV-Lip-DRV formulation enhances the relative delivery and retention of DRV in the brain. Due to technical difficulties with IN administration, only half the amount of DRV was used via IN compared to IP. If similar DRV concentrations are used with both routes the effect on DRV permeability to the brain using the IN route would be even more substantial.

To assess the off-target effect, the concentrations of DRV in the liver and lungs were also measured. No significant differences were observed between the DRV and EV-Lip-DRV groups in the liver for either administration route though the relative DRV concentration seems to be higher in the DRV group when administered via IP than IN route. Similarly in the lungs, there was a trend towards higher DRV concentration in the EV-Lip-DRV group via the IN group, though this did not reach statistical significance. Taken together, these results demonstrate that EV-Lip-DRV formulation using IN administration significantly increases drug distribution in the brain while reducing the off-target effects

Despite the advantages of IN administration, such as improved targeting and reduced systemic exposure, it is limited by the low volume that can be administered. Compared to the same IN dosing of PLGA-DRV in an earlier study, the EV-Lip-DRV showed a higher concentration of DRV and higher brain-to-plasma ratio in the brain at 3 h. These findings demonstrate that enhanced biocompatibility of the EV-Lip formulation may contribute to improved drug retention, reduced clearance, and more effective delivery to the brain. PLGA nanoparticles, while effective in drug delivery, often face challenges such as rapid clearance and potential immunogenicity. The disclosed EV-Lip-DRV formulation can address these issues by combining the biocompatibility of EVs with the stability and drug-loading capacity of liposomes, highlighting its potential superiority over only synthetic nanoparticles.

### Example 7

### Behavior Studies of DRV and EV-Lip-DRV in EcoHIV-Infected C57BL/6 Mice

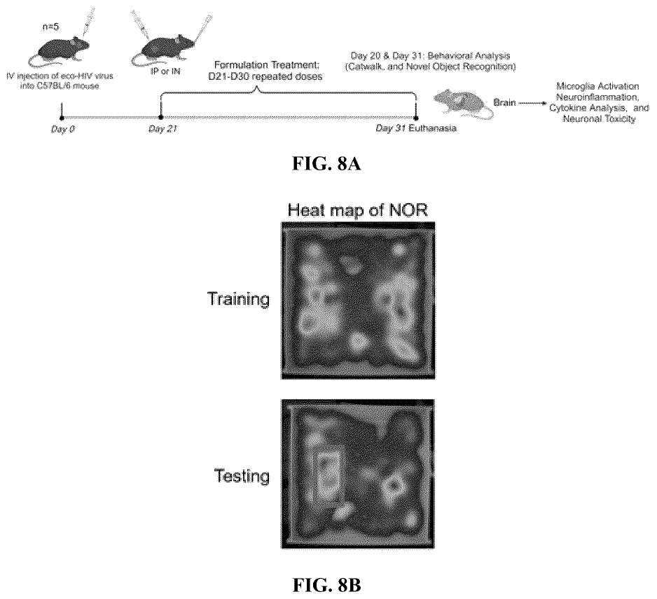

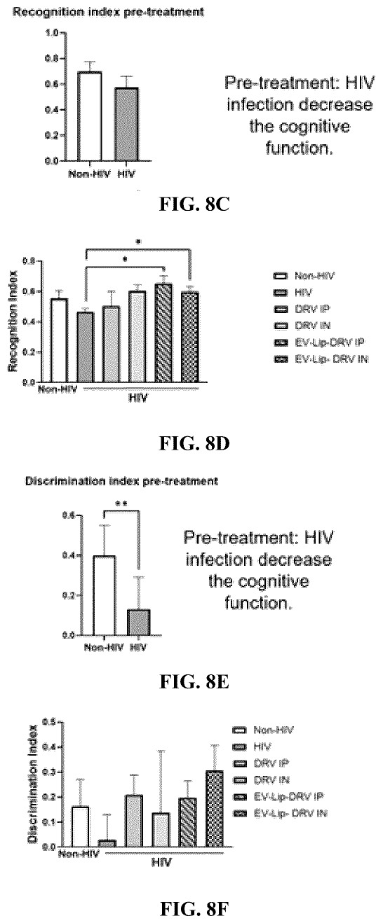

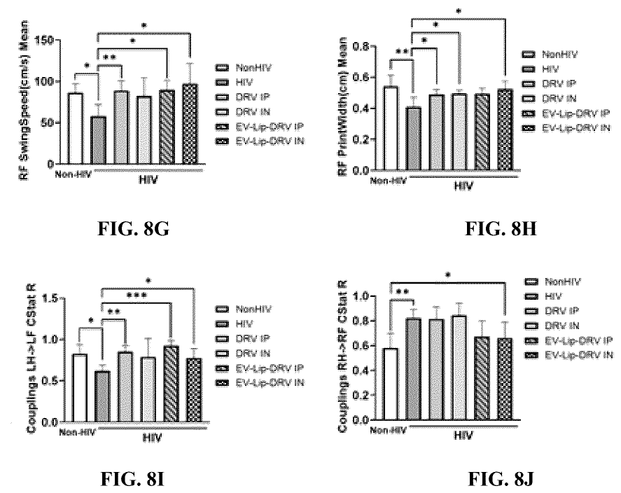

Behavioral studies were conducted to assess the motor and neurocognitive function of EcoHIV-infected C57BL/6 mice treated with 2.5 mg/kg of DRV or EV-Lip-DRV via intraperitoneal (IP) or intranasal (IN) administration for 10 days. The timeline of the study is depicted in FIG. 8A. Novel Object Recognition (NOR) and Catwalk gait analysis tests were performed to evaluate the treatment effects.

Cognitive Function—Novel Object Recognition (NOR) Test: FIG. 8B shows the results of the NOR test. Heat maps indicate the location of the new object during the testing phase. Pre-treatment assessments revealed a significant decline in cognitive function due to HIV infection, evidenced by lower recognition index (RI) and discrimination index (DI) (**p<0.01) values in HIV-infected mice compared to non-HIV controls. Post-treatment assessments demonstrated improvements in cognitive function for both DRV and EV-Lip-DRV groups using both administration routes.

Recognition Index (RI) (FIG. 8C and FIG. 8D): HIV-infected mice treated with EV-Lip-DRV via IP and IN administration showed significant improvement in RI compared to untreated HIV-infected controls (*p<0.05). However, DRV did not show a significant increase in RI using either route.

Discrimination Index (DI) (FIG. 8E and FIG. 8F): Both DRV and EV-Lip-DRV treatments, via IP and IN routes, showed a trend in improved DI in HIV-infected mice, aligning the DI values closer to those of the non-HIV control group but not statistically significant.

Overall, these results demonstrate that HIV infection impairs cognitive function, as evidenced by RI and DI, which to some extent is repaired by the treatment with EV-Lip-DRV, especially using IN administration.

Motor Function—Catwalk Gait Analysis: FIG. 8G, FIG. 8H, FIG. 8I and FIG. 8J present the Catwalk gait analysis results. Several parameters showed significant differences between uninfected and EcoHIV-infected and between control and treated groups in EcoHIV-infected mice. RF Swing Speed (FIG. 8G): HIV-infected mice showed a reduced right front (RF) swing speed compared to non-HIV control. Treatment with DRV and EV-Lip-DRV via both IP and IN routes significantly improved RF swing speed, with notable increases. However, there was no difference between DRV alone and EV-Lip-DRV or between the two routes of administration. RF Print Width (FIG. 8H): The HIV-infected group exhibited a narrower RF print width compared to non-HIV control. Both DRV (IP & IN) and EV-Lip-DRV (IN) treatments led to a significant increase in RF print width, indicating improved motor function. HIV-infected mice displayed altered couplings between left hind-left front (LH-LF) and right hind-right front (RH-RF) limbs, with significant deviations from the non-HIV control group (FIG. 8I and FIG. 8J). Treatments with DRV (IP) and EV-Lip-DRV (IP & IN) normalized left hind-left front (LH-LF) couplings (FIG. 8I), bringing them closer to control values with significant improvement observed, which was similar to the observations in RF Swing Speed. Only EV-Lip-DRV via IN significantly improved the coupling of right hind-right front (RH-RF) limbs (FIG. 8J). These results demonstrate that HIV infection impairs motor function, as evidenced by changes in RF swing speed, RF print width, and limb couplings. Treatment with DRV and EV-Lip-DRV effectively ameliorates these motor deficits, demonstrating the potential of both DRV and formulation to restore motor function in HIV-infected mice, especially via the IN route.

With the progression of HIV, patients have a range of cognitive, motor, and behavioral symptoms, which are hallmarks of HAND. The NOR test and Catwalk gait analysis were employed to evaluate cognitive and motor functions in HIV-infected mice, respectively. The findings from the NOR test demonstrate that HIV-infected mice have reduced cognitive function which to some extent is recovered by the treatment group, especially with EV-Lip-DRV using the IN route. The Catwalk gait analysis reveals impairments in motor function by HIV infection, which to some extent is recovered by EV-Lip-DRV treatment. Although Catwalk is not a typical test for HAND, it is used to measure movement disorders in Parkinson's disease (PD) and Huntington's disease (HD). As Catwalk measures motor, emotional, and cognitive deficits in HD, which are also characteristics of HAND, the test was used to measure motor and cognitive functions in HIV. Thus, in addition to NOR, the CatWalk test can also be used to study behavior deficits in HIV mice. Taken together these findings demonstrate that a daily dose of EV-Lip-DRV via IN route may offer a potentially novel therapeutic approach for managing neurocognitive and motor dysfunctions associated with HIV.

Notably that there is no perfect animal model to study HAND. The EcoHIV mouse model was selected mainly because it is a well-established HIV mouse model featuring behavioral deficits and neuropathological changes upon infection. Various behavior studies indicate cognitive (NOR, Morris Water Maze, Barnes Maze, Contextual and Cued Fear Conditioning, etc.), and motor (Rotarod Test, Grip Strength Test, Ladder Rung Walking Test, etc.) functions that may be used to study HAND in HIV mice. Nose-to-brain delivery has been explored for HIV treatment however, most of those studies focused on the improvement of brain drug concentration and its effect on HIV biomarkers. These results show the improvement in cognition and motor functions in HIV mice by EV-Lip formulation using the IN route.

### Example 8

### Effect of EV-Lip-DRV on Neuroinflammation, Neuronal Damage, and Oxidative Stress in EcoHIV Mice

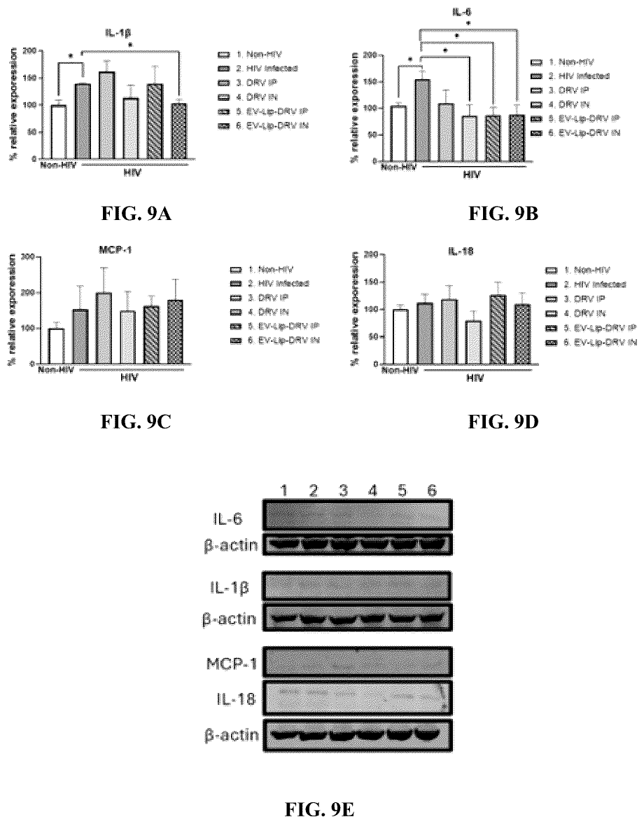

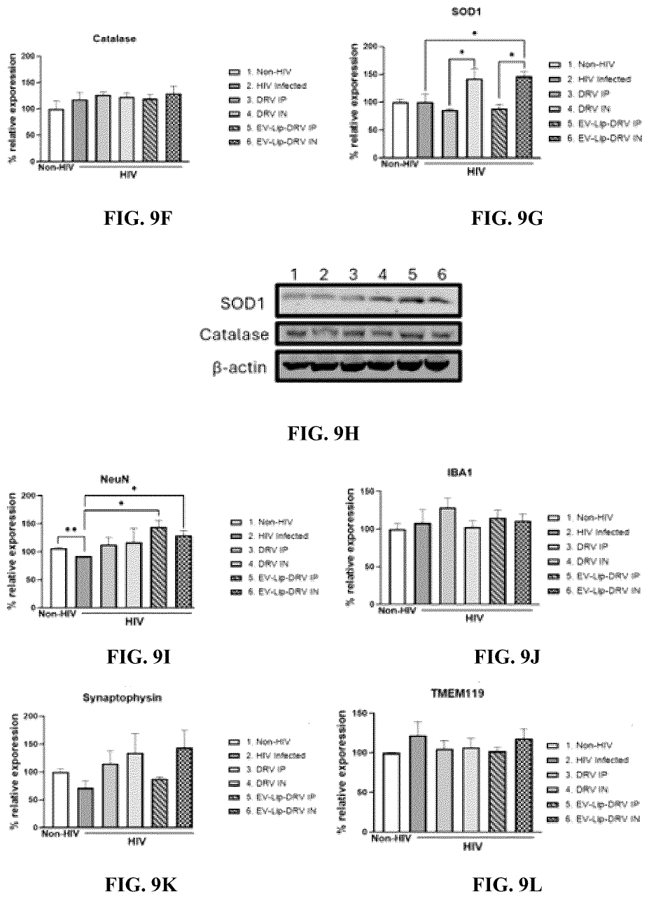

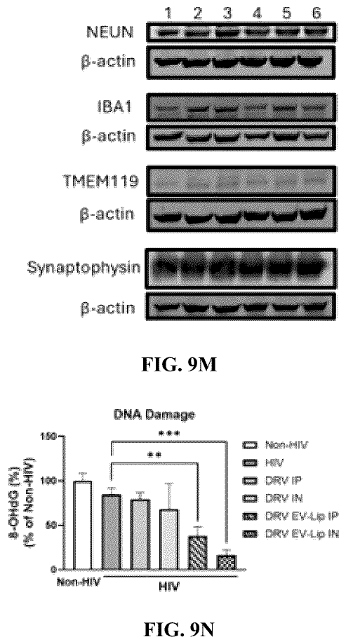

The protein expression of inflammatory cytokines and chemokines, oxidative stress markers, and neuronal markers in mice brains with and without EcoHIV infection was analyzed. FIG. 9E presents the western blot results for representative inflammatory cytokines IL-1β, IL-6, IL-18, and the chemokine MCP-1 in the brains of HIV-infected mice. As expected, HIV infection significantly elevated IL-1β and IL-6 levels (FIG. 9A and FIG. 9B). Treatment with EV-Lip-DRV via IN administration significantly reduced IL-1β and IL-6 levels (*p<0.05) (FIG. 9A and FIG. 9B). Although DRV via IP administration also reduced IL-1β levels, the change was not statistically significant (FIG. 9A). DRV via the IN route and EV-Lip-DRV via the IP route also significantly reduced IL-6 levels (*p<0.05) (FIG. 9B). No significant changes in MCP-1 and IL-18 levels were observed in the HIV-infected compared to the uninfected group, and among the different treatment groups compared to the non-treated group in EcoHIV (FIG. 9C and FIG. 9D).

FIG. 9H shows the western blot results for oxidative stress markers, catalase, and SOD1. HIV infection increased SOD1 levels, indicating elevated oxidative stress. Treatment with DRV and EV-Lip-DRV via IN administration significantly reduced SOD1 levels (*p<0.05) (FIG. 9G). However, catalase levels remained similar across all groups, demonstrating no significant changes in this oxidative stress marker (FIG. 9F).

FIG. 9M displays the western blot results for neuronal markers (NeuN, synaptophysin), and microglia markers (IBA1 and TMEM119). NeuN is a nuclear protein expressed in most neuronal cells indicating differentiation of neuron. IBA1 and TMEM119 are microglia markers indicating microglia activation and surface protein respectively. Synaptophysin serves as a marker for synapses, which is used to study synaptogenesis, synaptic density, and changes in synaptic connections in various neurological conditions. HIV infection significantly reduced NeuN levels compared to non-HIV control (**p<0.01) (FIG. 9I). Treatment with EV-Lip-DRV via both IP and IN significantly increased NeuN levels (*p<0.05) (FIG. 9I). No significant changes were observed in IBA1 and TMEM119 levels among the different groups (FIG. 9J and FIG. 9L). Although synaptophysin levels were decreased in HIV-infected mice and showed an increase in the treatment groups, these changes were not statistically significant (FIG. 9K).

The extent of DNA damage was evaluated by measuring 8-OHdG levels, a marker of DNA oxidation, in the brains of mice. As shown in FIG. 9D, treatment with EV-Lip-DRV, particularly via IP and IN administration, significantly reduced 8-OHdG levels compared to untreated HIV-infected mice (**p<0.01, ***p<0.001), indicating a reduction in DNA damage. However, no significant change was observed in DRV treatment regardless of the routes.

As noted before, the EV-Lip-DRV formulation modulates the cytokine and chemokine profile, decreasing pro-inflammatory markers (TNF-α, IL-1β, IL-6, IL-18, IL-8) without affecting anti-inflammatory cytokines (IL-10, IL-1RA) in U1 macrophages. The reduction of TNF-α, IL-1β, IL-6, and IL-18 in EV-Lip treatment in vitro may be caused by the activation of NF-κB pathway as the study showed MSC-derived EV can mitigate the overexpression of inflammatory cytokines in the mice liver cell line. Decreased levels of IL-6 was observed in DRV IN and EV-Lip-DRV of both routes (IP and IN) in HIV mice brains. The inflammatory response in the brain has been strongly correlated with cognitive impairment in EcoHIV mice. Modulating the levels of inflammatory cytokines and chemokines is one of the mechanisms by which the EV-Lip-DRV formulation mitigates HAND.

In the context of HIV infection, chronic inflammation and viral proteins contribute to increased ROS production, leading to neuronal damage and apoptosis. This oxidative damage, combined with neuroinflammation, exacerbates the neurocognitive decline observed in HAND patients. In the present disclosure, EV-Lip-DRV treatment increased SOD1 expression demonstrating that EV-Lip-DRV can protect the cells from oxidative damage in EcoHIV mice brain. This notion is further strengthened by the present observation that EV-Lip-DRV reduces oxidative DNA damage in EcoHIV mice. In addition, the in vitro data with U1 macrophages in which EV-Lip-DRV treatment reduces ROS level is consistent with in vivo study with EcoHIV mice where the treatment reduces oxidative stress/DNA damage. Together, these findings demonstrate a decrease in oxidative stress upon treatment with EV-Lip-DRV in EcoHIV mice.

HIV is known to reduce the levels of NeuN. This example also shows a decrease in NeuN in EcoHIV (FIG. 9C). More importantly, increased levels of NenN upon EV-Lip-DRV treatment demonstrates recovery of neuronal differentiation from HIV infection.

Gene expression of TMEM119 is significantly downregulated after homeostasis is disturbed in brain. This study did not significantly alter the level of synaptophysin either by HIV infection or by drug treatment. HIV infection appears to decrease its level, while EV-Lip-DRV treatment appears to rescue the decrease caused by HIV infection, demonstrating that EV-Lip-DRV may help recover neuronal damage induced by HIV.

### Example 9

### Membrane Fusion Study

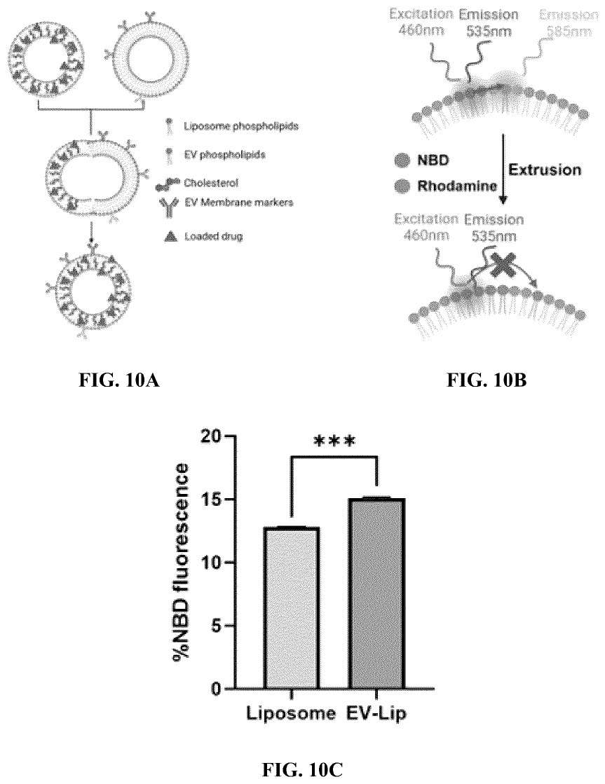

The membrane fusion between EVs and liposomes (FIG. 10A) was analyzed using FRET, as depicted in FIG. 10B. As shown in FIG. 10B, in liposomes alone Rhodamine quenched the NBD fluorescence. However, upon membrane fusion between EV and lipid membranes, there was an increase in NBD fluorescence. Although an exact percentage of membrane fusion could not be quantified, a significant increase in NBD fluorescence demonstrates successful membrane fusion (FIG. 10C).

### Example 10

### Development and Efficacy of EV-LIP-EVG+CUR Hybrid Nanoparticles

This example describes the formulation, characterization, and in vivo evaluation of a hybrid nanoparticle system co-encapsulating the integrase strand transfer inhibitor elvitegravir (EVG) and the pharmacological adjuvant curcumin (CUR). The EV-LIP-EVG+CUR combo nanoparticles were synthesized by fusing synthetic liposomes, comprising phosphatidylcholine, cholesterol, and DSPE-PEG, with plasma-derived extracellular vesicles. The hybrid vesicles were loaded with EVG and CUR using a thin-film hydration method followed by extrusion. Physicochemical analysis revealed that the combo nanoparticles possessed a mean hydrodynamic diameter of 187±nm via dynamic light scattering and a surface charge of −9.4±0.2 mV. Transmission electron microscopy confirmed a spherical core-shell morphology with a diameter of approximately 96±4 nm. Notably, the co-loading strategy achieved high encapsulation efficiencies of 63.6% for EVG and 62.59% for CUR, significantly outperforming solo-drug formulations.

The combo nanoparticles exhibited a biphasic drug release profile, characterized by an initial burst release followed by sustained delivery up to 96 hours. Under storage conditions at both room temperature and 4° C., the nanoparticles remained stable for at least 70 days, maintaining consistent size and drug retention. In vivo studies using a mouse model demonstrated that intranasal administration of the combo nanoparticles significantly increased drug delivery to the brain compared to free drug administration. At 1-hour post-administration, brain EVG levels reached 31-33 ng/g, representing a twofold increase in bioavailability. This route successfully bypassed the blood-brain barrier while minimizing accumulation in the liver, thereby reducing potential peripheral toxicity.

The therapeutic impact of the EV-LIP-EVG+CUR system was assessed in EcoHIV-infected mice, where the treatment effectively suppressed macrophage activation and neuroinflammation. Furthermore, the formulation reduced oxidative DNA damage, as evidenced by a decrease in 8-oxo-dG markers. Behavioral assays confirmed that the combo formulation rescued HIV-associated deficits, as treated mice showed a significant increase in the discrimination index during novel object recognition tests, indicating restored recognition memory. Spatial learning and memory were normalized in the Morris Water Maze to levels comparable to non-infected controls, and motor coordination was significantly improved as measured by CatWalk gait analysis.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

The foregoing written specification and following examples are considered to be sufficient to enable one skilled in the art to practice the invention. Various modifications of the invention in addition to those shown and described herein will become apparent to those skilled in the art from the foregoing description and following examples and fall within the scope of the appended claims. The disclosures of all citations in the specification are expressly incorporated herein by reference.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A nanoparticle composition comprising:
a hybrid vesicle formed by fusion of a lipid bilayer of a synthetic liposome and a membrane derived from a plasma-derived extracellular vesicle; and
an antiretroviral agent suitable for the treatment of human immunodeficiency virus (HIV) encapsulated within the hybrid vesicle,
wherein the synthetic liposome comprises phosphatidylcholine and cholesterol; and
wherein the hybrid vesicle has a diameter of less than 200 nm, the hybrid vesicle retains extracellular vesicle protein markers selected from the group consisting of CD63, CD9 and Alix, and the hybrid vesicle configured to deliver the antiretroviral agent across the blood-brain barrier following intranasal administration.

2. The composition of claim 1, wherein the wherein the phosphatidylcholine comprises 1,2-dioleoyl-sn-glycero-3-phosphocholine (DOPC).

3. The composition of claim 1, wherein the antiretroviral agent is a protease inhibitor.

4. The composition of claim 3, wherein the protease inhibitor is darunavir.

5. The composition of claim 1, wherein the antiretroviral agent is an integrase inhibitor.

6. The composition of claim 5, wherein the integrase inhibitor is elvitegravir.

7. The composition of claim 1, further comprising a pharmacological adjuvant encapsulated within the hybrid vesicle.

8. The composition of claim 7, wherein the pharmacological adjuvant is curcumin.

9. The composition of claim 1, wherein the encapsulation efficiency of the antiretroviral agent is at least 40%.

10. The composition of claim 1, wherein the composition exhibits sustained release of the antiretroviral agent over a period of at least 24 hours.

11. A method of treating HIV-associated neuropathogenesis in a subject in need thereof, the method comprising intranasally administering to the subject an effective amount of the composition of claim 1.

12. The method of claim 11, wherein the HIV-associated neuropathogenesis comprises HIV-associated neurocognitive disorder (HAND).

13. The method of claim 11, wherein administration of the nanoparticle composition reduces HIV replication in macrophages within the central nervous system.

14. The method of claim 11, wherein administration of the nanoparticle composition reduces neuroinflammation in brain tissue.

15. The method of claim 11, wherein administration of the nanoparticle composition reduces oxidative stress and neuronal damage in brain tissue.

16. The method of claim 11, wherein administration of the nanoparticle composition improves cognitive or motor function relative to administration of the antiretroviral agent alone.

17. A method of enhancing delivery of an antiretroviral agent to the brain, the method comprising intranasally administering to a subject a hybrid extracellular vesicle-liposome nanoparticle comprising the antiretroviral agent encapsulated therein, wherein the hybrid nanoparticle has a diameter of less than 200 nm and provides increased brain exposure of the antiretroviral agent relative to administration of the antiretroviral agent in non-encapsulated form.
